(19) United States
(12) Patent Application Publication
(10) Pub. No.: US 2018/0250420 A1
(43) Pub. Date: Sep. 6, 2018

(54) MULTIPLE HUMAN ANTIBODY-NANOPARTICLE CONJUGATES AND METHODS OF FORMATION

(71) Applicant: THE CURATORS OF THE UNIVERSITY OF MISSOURI, Columbia, MO (US)

(72) Inventors: Raghuraman KANNAN, Columbia, MO (US); Ajit Prakash ZAMBRE, Columbia, MO (US); Anandhi UPENDRAN, Columbia, MO (US); Henry WHITE, Columbia, MO (US)

(21) Appl. No.: 15/525,563

(22) PCT Filed: Nov. 13, 2015
(86) PCT No.: PCT/US2015/060654

§ 371 Date: May 9, 2017

Related U.S. Application Data
(60) Provisional application No. 62/123,350, filed on Nov. 13, 2014.

(51) Int. Cl. A61K 47/69; G01N 33/574; A61K 47/55; G01N 33/531; G01N 33/543; A61K 9/14; G01N 33/553; G01N 33/58; A61P 35/00

(52) U.S. Cl. CPC A61K 47/6923; G01N 33/57415; A61K 47/55; G01N 33/531; A61P 35/00; A61K 9/14; G01N 33/553; A61K 47/6925; G01N 33/587; G01N 33/54346

(57) ABSTRACT

A nanoconjugate that includes multiple antibody agents bonded to a single nanoparticles via a linker to form a conjugate having either electrostatic or covalent bonding or that retains original properties of the multiple antibody types prior to formation of the conjugate. Preferred methods provide for multiple antibody types attached to a single nanoparticle via electrostatic attachment, covalent or mixed covalent and electrostatic attachment.

### PRIORITY CLAIM AND REFERENCE TO RELATED APPLICATION

This application claims priority under 35 U.S.C 119 and all applicable statutes and treaties from prior U.S. Provisional Application Ser. No. 62/123,350, which was filed on Nov. 13, 2014.

### FIELD

A field of the invention is nanoparticle conjugate fabrication and the fabrication of testing, imaging and treatment using nanoparticle conjugates. Applications of the invention include the field of diagnosis, imaging and therapy of human cancer. Example specific applications of the invention include treatment of breast cancer.

### BACKGROUND

Currently accepted diagnoses for breast cancer start with a mammogram screening test, during which an x-ray image of a breast is taken to check for tumors.

Another technique for breast cancer diagnosis is ultrasound imaging. This technique provides a complementary image to that obtained by x-ray imaging.

Other techniques for diagnosing breast cancer include obtaining sample(s) of a suspected volume using needle aspiration for pathology analyses, x-ray computerized axial tomography (CAT) scan, and magnetic resonance imaging (MRI).

Post-diagnosis treatments include heightened monitoring and observation, surgical removal of identified tumors, removal of one or more breasts, external-beam radiation therapy, chemotherapy, and brachy therapy. Each of these techniques has some drawbacks, and the shortened life-time and fatality rate for breast cancer remains unacceptably high. There remains a need for new and improved therapeutic modalities, especially for treating inoperable breast cancer.

Typical chemotherapy agents include a stable pharmaceutical formulation comprising pertuzumab, trastuzumab, or other anti-HER2 human antibody agents. These agents deliver a low dose of antibody drug, administered over a period of several months. Difficulties include efficacy for certain tumors, and ability to accurately control dosage.

Human antibody agents have an affinity for human cancer cells.

Human antibody agents have an affinity for cancer vasculature.

Results contained in Katti et al., U.S. Published Patent Application Number 20120134918, show that a suitable pharmaceutical mixture containing Gum Arabic coated ¹⁹⁸Au radioactive gold nanoparticles is an effective theranostic agent for treatment of human prostate cancer. The Gum Arabic serves as a stabilizer. Results also show Gum Arabic coated non-radioactive gold (¹⁹⁷Au) are suitable theranostic human cancer agents.

Gold-198, also denoted herein and also in the literature as ¹⁹⁸Au, or as Au-108, because of its higher energy of emission (βₘₐₓ=0.96 MeV; half-life of 2.7 days) has been used as a permanent implant either alone or as an adjunct to external-beam radiation therapy of cancers. See, e.g., Knight P J, et al., “The use of Interstitial Radiation Therapy in the Treatment of Persistent, Localized, and Unresectable Cancer in Children,” Cancer 57: 951-4 (1986); Rich T. A., “Radiation Therapy for Pancreatic Cancer: Eleven Year Experience at the JCRT,” Int J Radiat Oncol Biol Phys. 11:759-63 (1985). Brachytherapy implants of large radioactive gold seeds provide rapid delivery of radiation at a very high dose rate, thus avoiding some of the radiologic problems associated with iodine. However, because of the high heterogeneity of radioactive ¹⁹⁸Au seeds and liquids, oncologists have developed a consensus that a majority of patients receiving low/high energy brachy therapy will experience post treatment symptoms including adverse side effects to severe clinical complications. Recognized complications for prostate cancer include proctitis, cystitis, incontinence and rectal bleeding. See, e.g., Dall'Era M A, et al, “Hyperbaric Oxygen Therapy for Radiation Induced Proctopathy in Men Treated for Prostate Cancer,” J Urol 176: 87-90 (2006).

The therapeutic nature of Gum Arabic coated ¹⁹⁸Au nanoparticles is due to emission of an energetic electron during radioactive decay of ¹⁹⁸Au (beta decay, where the maximum energy of emission (βₘₐₓ)=0.96 MeV. The half-life of ¹⁹⁸Au=2.7 days). A gamma ray is also emitted by ¹⁹⁸Au during beta decay. The gamma ray can be used for imaging, to obtain information on location and concentration of Gum Arabic ¹⁹⁸Au nanoparticles. ¹⁹⁸Au nanoparticles can be employed to produce a theranostic agent for cancer treatment.

The therapeutic nature of Gum Arabic coated radioactive ¹⁹⁹Au is also due to its emission of an energetic electron during radioactive decay (beta decay, where the maximum energy of emission (βₘₐₓ)=0.452 MeV. The half-life of ¹⁹⁹Au=3.13 days). A gamma ray is also emitted during beta decay. The gamma rays can be used for imaging, to obtain information on location and concentration of Gum Arabic coated ¹⁹⁹Au nanoparticles. The isotope ¹⁹⁹Au emits a 158 KeV gamma ray for scintigraphic imaging properties that is superior to the 412 KeV gamma ray emitted by ¹⁹⁸Au. ¹⁹⁹Au nanoparticles can be employed to produce a theranostic agent for cancer treatment.

Non-radioactive nanoparticles have been investigated for target specificity and increased retention for significant improvement in the treatment of the prostate and various inoperable tumors. See for example, Raghuraman Kannan et al, “Functionalized radioactive gold nanoparticles in tumor therapy” WIRES Nanomed Nanobiotechnol 2012, 4:42-51, doi: 10.1002/wnaan.161, for validation of the hypothesis that Gum Arabic-functionalized radioactive gold nanoparticles have high affinity toward cancer.

Artisans have also explored methods to produce antibody conjugated nanoparticles. An example is Hainfeld, U.S. Pat. No. 8,033,977. Hainfeld '977 discloses metal core particles, including gold particles, surrounded by a surface or shell layer of another material, such as molecules containing sulfur, phosphorous, amines or molecules with a thiol group. The shell layer can also include proteins, antibodies and fragments, or these can be linked. The technique for fabricating the targeted nanoparticles is during the synthesis of the particles, which can affect the targeting moiety or ligand. The Hainfeld antibodies are of the same type, and there is no recognition of any ability to have more than one type of antibody.

### SUMMARY OF INVENTION

Preferred embodiments provide nanoparticle conjugates for cancer diagnostic, imaging and therapy, with a nanoconjugate that includes multiple antibody types bonded to a single nanoparticles via a linker to form a conjugate having either electrostatic or covalent bonding and that retains original properties of the multiple antibody types prior to formation of the conjugate. The antibody agents may be human or animal antibody, antibody fragment, affibody, a small molecule, a recombinant humanized monoclonal antibody, or anti-hapten antibody. The nanoparticle may be a metallic or non-metallic nanoparticle, but is preferably a gold nanoparticle (AuNP). Metallic elements that can be used include, but are not limited to, platinum and palladium. Non-metallic nanoparticles can also be used. Polymeric or other non-metallic nanoparticle method of formation will follow the process with the methods of attachment via linkers and selection of simultaneous or sequential covalent and/or electrostatic attachment. The linker between the antibody agents and said nanoparticle includes thiol and ethylene glycol. The exemplary conjugates include electrostatic bonded pertuzumab—AuNP conjugates, covalent bonded pertuzumab—AuNP conjugates, electrostatic bonded trastuzumab—AuNP conjugates, and covalent bonded trastuzumab—AuNP conjugates.

Preferred embodiments also provide a method for forming multiple human antibody type nanoparticle conjugates. The methods retain properties of the multiple human antibody types with a fabrication process that also allows control of the bonding mode of the antibody to the surface of AuNP, such that the bonding mode can be predetermined to be either electrostatic or covalent.

### BRIEF DESCRIPTION OF THE DRAWINGS

FIG. 1 is a schematic diagram showing electrostatic and covalent processes of the invention for formation of dual antibody agent nanoconjugates of the invention via simultaneous and sequential linking;

FIG. 2 is a schematic diagram showing electrostatic and covalent processes of the invention for formation of dual antibody agent nanoconjugates of the invention via sequential electrostatic/electrostatic linking and electrostatic/covalent linking;

FIGS. 3A-3B are schematic diagrams showing preferred purification and isolation processes;

FIG. 4 plots HPLC chromatograms for the two dual antibody agent nanoconjugates prepared by simultaneous addition.

FIG. 5 plots HPLC chromatograms of experimental nanoconjugates prepared by sequential addition;

FIG. 6 includes schematic representations and TEM images of antibody nanoconjugates prepared by covalent and electrostatic conjugation via simultaneous addition;

FIG. 7 includes schematic representation and TEM images of antibody nanoconjugates prepared by covalent and electrostatic conjugation via sequential addition;

FIGS. 8A-8B plot UV-Vis spectra for dual antibody nanoconjugates prepared by covalent and electrostatic linking via simultaneous and sequential addition

FIGS. 9A-9B include measured physiochemical values for characterization of dual antibody nanoconjugates prepared by covalent and electrostatic linking via simultaneous and sequential addition

FIGS. 10A-10B are schematic representations of dual antibody gold nanoconjuates for evaluation of antibody by Bradford assay processes (listed at bottom of each panel)

FIGS. 11A-11B include protein estimation values by Bradford assay for dual antibody nanoconjugates prepared via sequential and simultaneous addition and linked via covalent and electrostatic conjugation

FIG. 12 is a schematic illustration of a purification and isolation process utilized for proteomics analysis for four example isolated nanoconjugates;

FIG. 13 includes histograms showing average spectral counts for both heavy and light chains for dual antibody conjugates obtained by simultaneous addition;

FIG. 14 includes histograms showing exclusive spectral counts for both heavy and light chains for dual antibody conjugates obtained by simultaneous addition;

FIG. 15 includes histograms showing % sequence coverage for both heavy and light chains for \ dual antibody conjugates obtained by simultaneous addition;

FIG. 16 includes histograms showing average spectral counts for both heavy and light chains for dual antibody conjugates obtained by sequential addition;

FIG. 17 includes histograms showing exclusive spectral counts for both heavy and light chains for dual antibody conjugates obtained by sequential addition;

FIG. 18 includes values in the histogram showing % sequence coverage for both heavy and light chains for dual antibody conjugates obtained by sequential addition;

FIG. 19 includes values, shown as histogram bars, for total spectral count, exclusive spectral count, and % sequence coverage values obtained for pertuzumab heavy chain and pertuzumab light chain example nanoconjugates;

FIG. 20 includes values, shown as histogram bars, for total spectral count, exclusive spectral count, and % sequence coverage obtained for trastuzumab heavy chain and pertuzumab light chain example nanoconjugates;

FIG. 21 includes values, shown as histogram bars, for total spectral count, exclusive spectral count, and % sequence coverage, represented as histogram bars, for each of the following: pertuzumab heavy chain, pertuzumab light chain, trastuzumab heavy chain, and trastuzumab light chain example nanoconjugates;

FIG. 22 includes (Total) spectral count, exclusive (spectral count) and % sequence coverage for each of the following: pertuzumab heavy chain and light chain, and for traztuzumab heavy chain and light chain.

FIG. 23 includes (Total) spectral count, exclusive (spectral count) and % sequence coverage for the dual antibody gold nanoconjugates prepared by covalent and electrostatic conjugation both by simultaneous and sequential addition.

FIG. 24 In vitro cytotoxicity studies in breast cancer SkBR3 and MCF7 cells when pertuzumab and/or trastuzumab gold nanoconjugates obtained by electrostatic and covalent conjugation were incubated for 96 hrs and analysed by MTT assay.

FIG. 25 A graphical representation of in vitro cytotoxicity studies in breast cancer SkBR3 and MCF7 cells when pertuzumab and/or trastuzumab gold nanoconjugates obtained by electrostatic and covalent conjugation were incubated for 96 hrs and analysed by MTT assay.

FIG. 26 In vitro cytotoxicity studies in breast cancer SkBR3 and MCF7 cells when pertuzumab and trastuzumab (Dual antibody) gold nanoconjugates obtained by electrostatic and covalent conjugation were incubated for 96 hrs and analysed by MTT assay.

FIG. 27 A graphical representation of comparison of in vitro cytotoxicity studies in breast cancer SkBR3 and MCF7 cells when pertuzumab and trastuzumab (Dual antibody) gold nanoconjugates obtained by electrostatic and covalent conjugation were incubated for 96 hrs and analysed by MTT assay.

FIG. 28 Western blot analysis of pertuzumab and trastuzumab (Dual antibody) gold nanoconjugates obtained by electrostatic and covalent conjugation in breast cancer SkBR3 cells analyzing the expression of proteins, HER2, phosphor-HER2, HER3, phospho-HER3, Akt, phospho-Akt, ERK, phospho-ERK, MEK, phospho-MEK, and actin.

FIG. 29 Western blot analysis of pertuzumab and trastuzumab (Dual antibody) gold nanoconjugates obtained by electrostatic and covalent conjugation in breast cancer SkBR3 cells analyzing the expression of proteins, HER2, phospho-HER2.

FIG. 30 Western blot analysis of pertuzumab and trastuzumab (Dual antibody) gold nanoconjugates obtained by electrostatic and covalent conjugation in breast cancer SkBR3 cells analyzing the expression of proteins, HER3, phospho-HER3.

FIG. 31 Western blot analysis of pertuzumab and trastuzumab (Dual antibody) gold nanoconjugates obtained by electrostatic and covalent conjugation in breast cancer SkBR3 cells analyzing the expression of proteins, AkT, phospho-AkT.

FIG. 32 Western blot analysis of pertuzumab and trastuzumab (Dual antibody) gold nanoconjugates obtained by electrostatic and covalent conjugation in breast cancer SkBR3 cells analyzing the expression of proteins, ERK1, ERK2, phospho-ERK.

FIG. 33 Western blot analysis of pertuzumab and trastuzumab (Dual antibody) gold nanoconjugates obtained by electrostatic and covalent conjugation in breast cancer SkBR3 cells analyzing the expression of proteins, MEK, and phospho-MEK.

FIG. 34 Western blot analysis of pertuzumab and trastuzumab (Dual antibody) gold nanoconjugates obtained by electrostatic and covalent conjugation in breast cancer SkBR3 cells analyzing the expression of proteins, Actin.

FIG. 35 Summary of western blot analysis of pertuzumab and trastuzumab (Dual antibody) gold nanoconjugates obtained by electrostatic and covalent conjugation in breast cancer SkBR3 cells analyzing the expression of proteins, HER2, phosphor-HER2, HER3, phospho-HER3, Akt, phospho-Akt, ERK, phospho-ERK, MEK, phospho-MEK, and actin.

FIG. 36 ELISA binding plots of experimental nanoconjugates.

### DETAILED DESCRIPTION OF THE INVENTION

Preferred embodiments of the invention provide biocompatible multiple human antibody agent nanoconjugates. The multiple antibody agent means the multiple different types of antibodies, or fragments, etc., are attached to the nanoparticle. The invention also includes particular ratios of each type of antibody agent and predetermination of therapeutic properties. The nanoconjugates include a nanoparticle, with two different antibody agents attached to the nanoparticle. In preferred embodiments, each of two different antibody agents is bonded covalently or electrostatically, and in other embodiments, one is bonded electrostatically and the other covalently. With selection of preferred antibody agents, preferred nanoconjugates provide therapeutic agent for the treatment of cancer, such as breast cancer. Selection of particular gold nanoparticles permits preferred nanoconjugates to serve as imaging agents, thereby acting as theranostic agents for imaging, therapy and diagnostic of human cancer.

Specific radioactive emitting and non-emitting gold nanoparticles have individually acted as imaging and therapy agents. Specific human antibodies have individually acted as therapy agents. However, singular agents with dual imaging and therapeutic (“theranostic”) capabilities that comprises a human antibody bonded to a gold nanoparticle have not been provided to the inventors' knowledge. The present inventors have recognized that a dual theranostic agent would provide tremendous consistency in the follow-up of therapy studies and could also minimize regulatory steps leading to final approval by the Food and Drug Administration (FDA). The inventors have also recognized that theranostic properties of a human antibody bonded to a gold nanoparticle offer realistic clinical possibilities for use as dual imaging, diagnostic and therapy agents

Preferred embodiments include Gum Arabic-functionalized radioactive and non-radioactive gold nanoparticles and human antibody agents. A preferred dual antibody conjugation includes pertuzumab and trastuzumab, which possess an affinity towards cancer. The Gum-Arabic gold nanoparticle of the nanoconjugate also provides affinity towards cancer. This dual affinity makes the affinity of the nanoconjugate independent of the type of gold nanoparticle, i.e., non-radioactive or radioactive. Other embodiments include citrate coated nanoparticles, and in such embodiments affinity is provide via the antibody agents, such as pertuzumab and trastuzumab.

Example experimental multiple type human antibody-AuNP conjugates formed demonstrate embodiments of the present invention were synthesized using non-radioactive 197Au.

The nature of the binding to cancer cells of human antibody-AuNP conjugates containing any and all admixture ratios of non-radioactive 197Au, radioactive 198Au, and radioactive 199Au are expected to be the same in pharmaceutical applications.

Preferred embodiments include dual human antibody—gold nanoparticle conjugates in a suitable pharmaceutical formulation wherein the gold nanoparticles are coated with citrate, and used in medical applications such as diagnosis, imaging, and therapy of cancers, including, but not limited to, human cancers, and wherein the therapy is by use of one or more of the techniques in the list including, but not limited to, chemotherapy, brachy therapy, and other techniques, and wherein the imaging is by use of one or more of the techniques in the list including, but not limited to, MRI, CAT, ultrasound and other techniques.

Similar chemical behavior and binding affinity to human cancer possessed by human antibodies and human antibody fragments and modified human antibodies, supports nanoconjugates of the invention as including nanoconjugates with antibody agents of human antibody fragments and modified human antibodies-gold nanoparticle conjugates (including non-radioactive and radioactive gold elements), for use in a suitable pharmaceutical formulation for use in medical applications such as diagnosis, imaging, and therapy of human cancers.

Preferred linkers for a nanoparticle conjugates for a gold nanoparticle and dual human antibodies of pertuzumab and trastuzumab possesses a molecular weight of 3400 Da.

Preferred linkers for a nanoparticle conjugates for a gold nanoparticle and dual human antibodies of pertuzumab and trastuzumab possesses a molecular weight of 2000 Da.

Antibody agents for nanoconjugates of the invention can take various forms.

For instance, they may be native antibodies, as naturally found in mammals. Native antibodies are made up of heavy chains and light chains. The heavy and light chains are both divided into variable domains and constant domains. The ability of different antibodies to recognize different antigens arises from differences in their variable domains, in both the light and heavy chains. Light chains of native antibodies in vertebrate species are either kappa (.kappa.) or lambda (.lambda.), based on the amino acid sequences of their constant domains. The constant domain of a native antibody's heavy chains will be .alpha., .delta., .epsilon., .gamma. or .mu., giving rise respectively to antibodies of IgA, IgD, IgE, IgG, or IgM class. Classes may be further divided into subclasses or isotypes e.g. IgG1, IgG2, IgG3, IgG4, IgA, IgA2, etc. Antibodies may also be classified by allotype e.g. a .gamma. heavy chain may have G1m allotype a, f, x or z, G2m allotype n, or G3m allotype b0, b1, b3, b4, b5, c3, c5, g1, g5, s, t, u, or v; a .kappa. light chain may have a Km(1), Km(2) or Km(3) allotype. A native IgG antibody has two identical light chains (one constant domain C.sub.L and one variable domain V.sub.L) and two identical heavy chains (three constant domains C.sub.H1 C.sub.H2 & C.sub.H3 and one variable domain V.sub.H), held together by disulfide bridges. The domain and three-dimensional structures of the different classes of native antibodies are well known.

Where an antibody agent for nanoconjugates of the invention has a light chain with a constant domain, it may be a .kappa. or .lambda. light chain (although, in some embodiments, antibodies must have a .lambda. light chain). Where an antibody of the invention has a heavy chain with a constant domain, it may be a .alpha., .delta., .epsilon., .gamma. or .mu. heavy chain. Heavy chains in the .gamma. class (i.e. IgG antibodies) are preferred. Antibodies of the invention may have any suitable allotype (see above).

Antibody agents for nanoconjugates of the invention may be fragments of native antibodies that retain antigen binding activity. For instance, papain digestion of native antibodies produces two identical antigen-binding fragments, called “Fab” fragments, each with a single antigen-binding site, and a residual “Fc” fragment without antigen-binding activity. Pepsin treatment yields a “F(ab′).sub.2” fragment that has two antigen-binding sites. “Fv” is the minimum fragment of a native antibody that contains a complete antigen-binding site, consisting of a dimer of one heavy chain and one light chain variable domain. Thus an antibody of the invention may be Fab, Fab′, F(ab′).sub.2, Fv, or any other type, of fragment of a native antibody. Antibodies of the invention may incorporate Dual-Affinity Re-Targeting (DART) platform technology, which is focused on dual specificity “antibody-like” therapeutic proteins capable of targeting multiple different epitopes with a single recombinant molecule.

Antibody agents for nanoconjugates of the invention may be a “single-chain Fv” (“scFv” or “sFv”), comprising a V.sub.H and V.sub.L domain as a single polypeptide chain. Typically the V.sub.H and V.sub.L domains are joined by a short polypeptide linker (e.g. .gtoreq.12 amino acids) between the V.sub.H and V.sub.L domains that enables the scFv to form the desired structure for antigen binding. A typical way of expressing scFv proteins, at least for initial selection, is in the context of a phage display library or other combinatorial library. Multiple scFvs can be linked in a single polypeptide chain.

Antibody agents for nanoconjugates of the invention may be a “diabody” or “triabody” etc., comprising multiple linked Fv (scFv) fragments. By using a linker between the V.sub.H and V.sub.L domains that is too short to allow them to pair with each other (e.g. <12 amino acids), they are forced instead to pair with the complementary domains of another Fv fragment and thus create two antigen-binding sites.

Antibody agents for nanoconjugates of the invention may be a single variable domain or VHH antibody. Antibodies naturally found in camelids (e.g. camels and llamas) and in sharks contain a heavy chain but no light chain. Thus antigen recognition is determined by a single variable domain, unlike a mammalian native antibody. The constant domain of such antibodies can be omitted while retaining antigen-binding activity. One way of expressing single variable domain antibodies, at least for initial selection, is in the context of a phage display library or other combinatorial library. Published reports discloses a camelid antibody (CC07) raised against a H5N2 strain of influenza A virus and having specificity for neuraminidase.

Antibody agents for nanoconjugates of the invention may be a “domain antibody” (dAb). Such dAbs are based on the variable domains of either a heavy or light chain of a human antibody and have a molecular weight of approximately 13 kDa (less than one-tenth the size of a full antibody). By pairing heavy and light chain dAbs that recognize different targets, antibodies with dual specificity can be made, dAbs are cleared from the body quickly, but can be sustained in circulation by fusion to a second dAb that binds to a blood protein (e.g. to serum albumin), by conjugation to polymers (e.g. to a polyethylene glycol), or by other techniques.

Antibody agents for nanoconjugates of the invention may be a chimeric antibody, having constant domains from one organism (e.g. a human) but variable domains from a different organism (e.g. non-human). Chimerization of antibodies was originally developed in order to facilitate the transfer of antigen specificity from easily-obtained murine monoclonal antibodies into a human antibody, thus avoiding the difficulties of directly generating human monoclonal antibodies. Because the inventor already provided human antibodies as a starting point for further work then chimerization will not typically be required for performing the invention. If non-human antibodies are generated, however, then they can be used to prepare chimeric antibodies. Similarly, if human antibodies of the invention are to be used in non-human organisms then their variable domains could be joined to constant domains from the non-human organism.

Antibody agents for nanoconjugates of the invention may be a CDR-grafted antibody. The CDR grafting process is described above. Because the inventor already provided human antibodies as a starting point for further work then, as for chimerisation, CDR grafting will not typically be required.

Thus the term “antibody agent” as used herein encompasses a range of proteins having diverse structural features (usually including at least one immunoglobulin domain having an all-.beta. protein fold with a 2-layer sandwich of anti-parallel .beta.-strands arranged in two .beta.-sheets), but all of the proteins possess the ability to bind to proteins.

Antibody agents for nanoconjugates of the invention may include a single antigen-binding site (e.g. as in a Fab fragment or a scFv) or multiple antigen-binding sites (e.g. as in a F(ab′).sub.2 fragment or a diabody or a native antibody). Where an antibody has more than one antigen-binding site then advantageously it can result in cross-linking of antigens.

Where an antibody has more than one antigen-binding site, the antibody may be mono-specific (i.e. all antigen-binding sites recognize the same antigen) or it may be multi-specific (i.e. the antigen-binding sites recognize more than one antigen). Thus, in a multi-specific antibody, at least one antigen-binding site will recognize a H5N1 influenza A virus and at least one antigen-binding site will recognize a different antigen.

Antibody agents for nanoconjugates of the invention may include a non-protein substance e.g. via covalent conjugation. For example, an antibody may include a radio-isotope e.g. the Zevalin™ and Bexxar™ products include .sup.90Y and .sup.131I isotopes, respectively. As a further example, an antibody may include a cytotoxic molecule e.g. Mylotarg™ is linked to N-acetyl-.gamma.-calicheamicin, a bacterial toxin. As a further example, an antibody may include a covalently-attached polymer e.g. attachment of polyoxyethylated polyols or polyethylene glycol (PEG) has been reported to increase the circulating half-life of antibodies.

In some embodiments of the invention, an antibody can include one or more constant domains (e.g. including C.sub.H or C.sub.L domains). As mentioned above, the constant domains may form a .kappa. or .lambda. light chain or an .alpha., .delta., .epsilon., .gamma. or .mu. heavy chain. Where antibody agent for nanoconjugates of the invention includes a constant domain, it may be a native constant domain or a modified constant domain. A heavy chain may include either three (as in .alpha., .gamma., .delta. classes) or four (as in .mu., .epsilon. classes) constant domains. Constant domains are not involved directly in the binding interaction between an antibody and an antigen, but they can provide various effector functions, including but not limited to: participation of the antibody in antibody-dependent cellular cytotoxicity (ADCC); C1q binding; complement dependent cytotoxicity; Fc receptor binding; phagocytosis; and down-regulation of cell surface receptors.

The constant domains can form a “Fc region”, which is the C-terminal region of a native antibody's heavy chain. Where an antibody of the invention includes a Fc region, it may be a native Fc region or a modified Fc region. A Fc region is important for some antibodies' functions e.g. the activity of Herceptin™ is Fc-dependent. Although the boundaries of the Fc region of a native antibody may vary, the human IgG heavy chain Fc region is usually defined to stretch from an amino acid residue at position Cys226 or Pro230 to the heavy chain's C-terminus. The Fc region will typically be able to bind one or more Fc receptors, such as a Fc.gamma.RI (CD64), a Fc.gamma.RII (e.g. Fc.gamma.RIIA, Fc.gamma.RIIB1, Fc.gamma.RIIB2, Fc.gamma.RIIC), a Fc.gamma.RIII (e.g. Fc.gamma.RIIIA, Fc.gamma.RIIIB), a FcRn, Fc.alpha.R (CD89), Fc.delta.R, Fc.mu.R, a Fc.epsilon.RI (e.g. Fc.epsilon.RI.alpha.beta.gamma.sub.2 or Fc.epsilon.RI.alpha.gamma.sub.2), Fc.epsilon.RII (e.g. Fc.epsilon.RIIA or Fc.epsilon.RIIB), etc. The Fc region may also or alternatively be able to bind to a complement protein, such as C1q. Modifications to an antibody's Fc region can be used to change its effector function(s) e.g. to increase or decrease receptor binding affinity. It has been reported that effector functions may be modified by mutating Fc region residues 234, 235, 236, 237, 297, 318, 320 and/or 322. Similarly, others report that effector functions of a human IgG1 can be improved by mutating Fc region residues (EU Index Kabat numbering) 238, 239, 248, 249, 252, 254, 255, 256, 258, 265, 267, 268, 269, 270, 272, 276, 278, 280, 283, 285, 286, 289, 290, 292, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 and/or 439. Modification of Fc residues 322, 329 and/or 331 is reported elsewhere for modifying C1q affinity of human IgG antibodies, and residues 270, 322, 326, 327, 329, 331, 333 and/or 334 are selected for modification in other reports. Mapping of residues important for human IgG binding to FcRI, FcRII, FcRIII, and FcRn receptors has also be reported together with the design of variants with improved FcR-binding properties. Whole C.sub.H domains can be substituted between isotypes e.g. others discloses antibodies in which the C.sub.H3 domain (and optionally the C.sub.H2 domain) of human IgG4 is substituted by the C.sub.H3 domain of human IgG1 to provide suppressed aggregate formation. It has also been reported that mutation of arginine at position 409 (EU index Kabat) of human IgG4 to e.g. lysine shows suppressed aggregate formation. Mutation of the Fc region of available monoclonal antibodies to vary their effector functions is known such as mutation studies for RITUXAN™ to change C1q-binding, and other reports include mutation studies for NUMAX™ to change FcR-binding, with mutation of residues 252, 254 and 256 giving a 10-fold increase in FcRn-binding without affecting antigen-binding.

Antibody agents for nanoconjugates of the invention will typically be glycosylated. N-linked glycans attached to the C.sub.H2 domain of a heavy chain, for instance, can influence C1q and FcR binding, with aglycosylated antibodies having lower affinity for these receptors. The glycan structure can also affect activity e.g. differences in complement-mediated cell death may be seen depending on the number of galactose sugars (0, 1 or 2) at the terminus of a glycan's biantennary chain. An antibody's glycans preferably do not lead to a human immunogenic response after administration.

Antibody agents for nanoconjugates of the invention can be prepared in a form free from products with which they would naturally be associated. Contaminant components of an antibody's natural environment include materials such as enzymes, hormones, or other host cell proteins.

Antibody agents for nanoconjugates of the invention can be used directly (e.g. as the active ingredient for pharmaceuticals or diagnostic reagents), or they can be used as the basis for further development work. For instance, an antibody may be subjected to sequence alterations or chemical modifications in order to improve a desired characteristic e.g. binding affinity or avidity, pharmacokinetic properties (such as in vivo half-life), etc. Techniques for modifying antibodies in this way are known in the art. For instance, an antibody may be subjected to “affinity maturation”, in which one or more residues (usually in a CDR) is mutated to improve its affinity for a target antigen. Random or directed mutagenesis can be used, but other reports describe affinity maturation by V.sub.H and V.sub.L domain shuffling as an alternative to random point mutation. Published reports show how NUMAX™ was derived by a process of in vitro affinity maturation of the CDRs of the heavy and light chains of SYNAGIS™, giving an antibody with enhanced potency and 70-fold greater binding affinity for RSV F protein.

Preferred multiple type antibody nanoconjugates of the invention are specific for Her2. The antibody agents will have a tighter binding affinity for that antigen than for an arbitrary control antigen e.g. than for a human protein. Preferred antibodies have nanomolar or picomolar affinity constants for target antigens e.g. 10.sup.-9 M, 10.sup.-10 M, 10.sup.-11 M, 10.sup.-12 M, 10.sup.-13 M or tighter). Such affinities can be determined using conventional analytical techniques e.g. using surface plasmon resonance techniques as embodied in BIAcore™ instrumentation and operated according to the manufacturer's instructions. Radio-immunoassay using radiolabeled target antigen (Her2) is another method by which binding affinity may be measured.

In preferred embodiments one or more of the antibody agents bonded to the AuNP may be at least one of the antibody in the list including, but not limited to, an antibody, an antibody fragment, affibody, a small molecule, a recombinant humanized monoclonal antibody, a rabbit antibody, a goat antibody, a mouse antibody, and an anti-hapten antibody.

Conjugates comprising multiple type human antibodies bonded to gold nanoparticles can be synthesized under clinical settings. This was demonstrated in experiments. The results will be discussed.

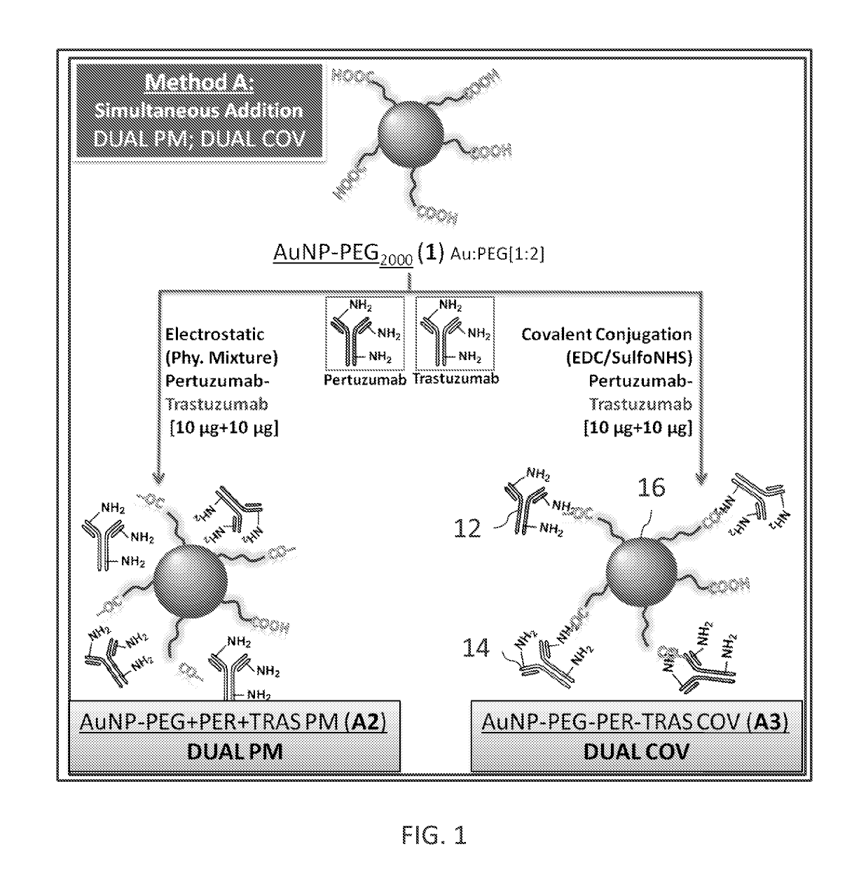

FIG. 1 illustrates a preferred method for dual human antibodies bonded 12 and 14 to gold nanoparticle 16, via simultaneous addition of both antibodies to prepare examples of AuNP-dual antibody conjugates; namely, AuNP-PEG+PER+TRAS PM (A2) by an electrostatic method and AuNP-PEG-PER-TRAS COV (A3) by a covalent method. Note that the abbreviated terms “DUAL PM”, Dual PM, and the end tag “PM” when formed by simultaneous addition, are used interchangeably thorough out the application and pertain to electrostatic bonding of both antibodies to a AuNP-PEG intermediate chemical entity for a conjugate formed by simultaneous addition method. Note that the abbreviated terms, “DUAL COV”, Dual COV, and the end tag “COV” when formed by simultaneous addition, may be used interchangeably thorough out the application and pertain to covalent bonding of both antibodies to a AuNP-PEG intermediate chemical entity for a conjugate formed by simultaneous addition method. Also note that the abbreviated terms “A” and “method A” are associated with the simultaneous addition method, whereas the abbreviated terms “B” and “method B” are associated with sequential addition method.

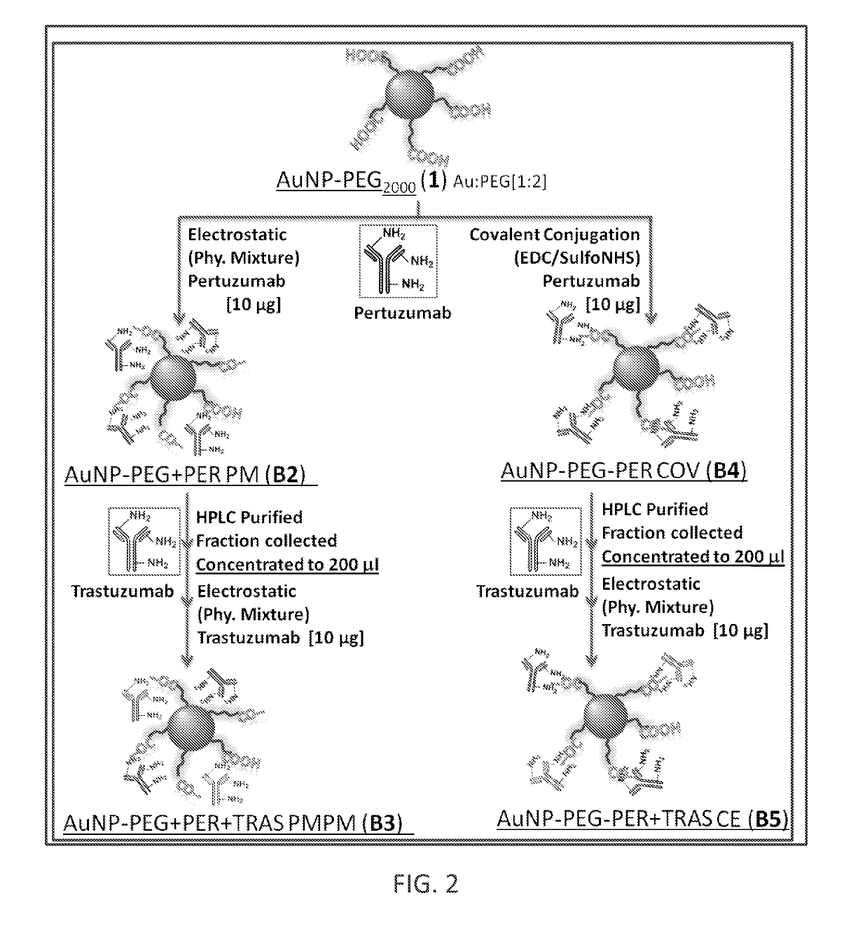

FIG. 2 illustrates on the left side sequential addition of antibodies to prepare the AuNP-dual antibody conjugate AuNP-PEG+PER+TRAS PMPM (B3) by a preferred electrostatic/electrostatic method. The schematic representation on the right side is sequential addition of antibodies to prepare the AuNP-dual antibody conjugate AuNP-PEG-PER-TRAS CE by a covalent method followed by electrostatic method. B2 and B4, shown schematically, represent intermediate chemical entities in formation of B3 and B5, respectively. The abbreviated terms, “PMPM” and “PM+PM”, associated with sequential addition, are used interchangeably throughout the application. The abbreviated terms “CE” and “COV+ELE”, associated with sequential addition, are used interchangeably throughout the application.

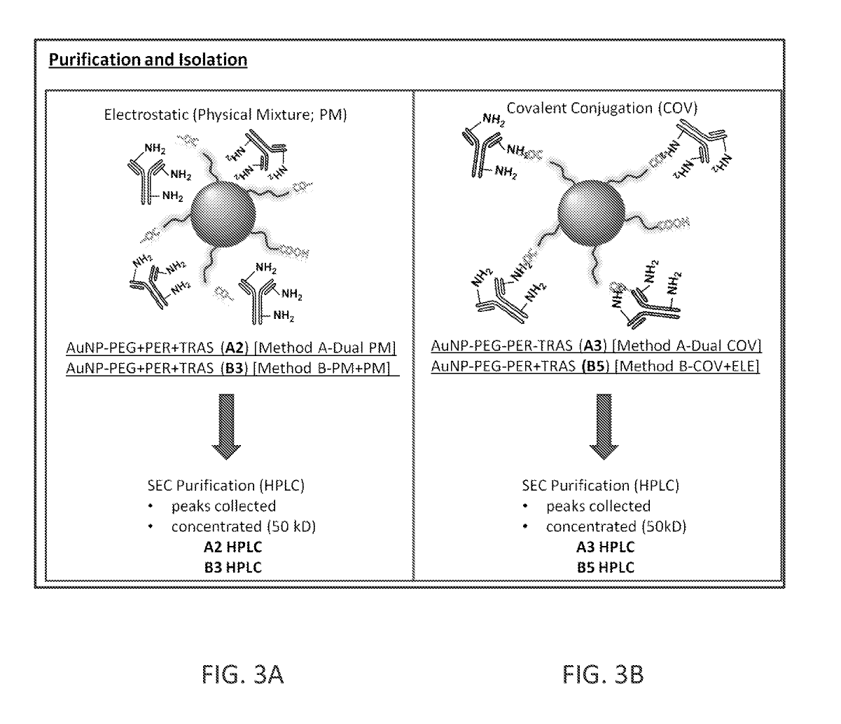

FIGS. 3A and 3B show preferred purification and isolation processes utilized in experiments to obtain AuNP—dual antibody conjugates AuNP-PEG+PER+TRAS Dual PM (A2) formed by simultaneous method and AuNP-PEG+PER+TRAS PM+PM (B3) formed by sequential method (FIG. 3A), and for conjugates AuNP-PEG-PER-TRAS Dual COV (A3) formed by simultaneous method and AuNP-PEG-PER+TRAS COV+ELE (B5) formed by sequential method (FIG. 3B).

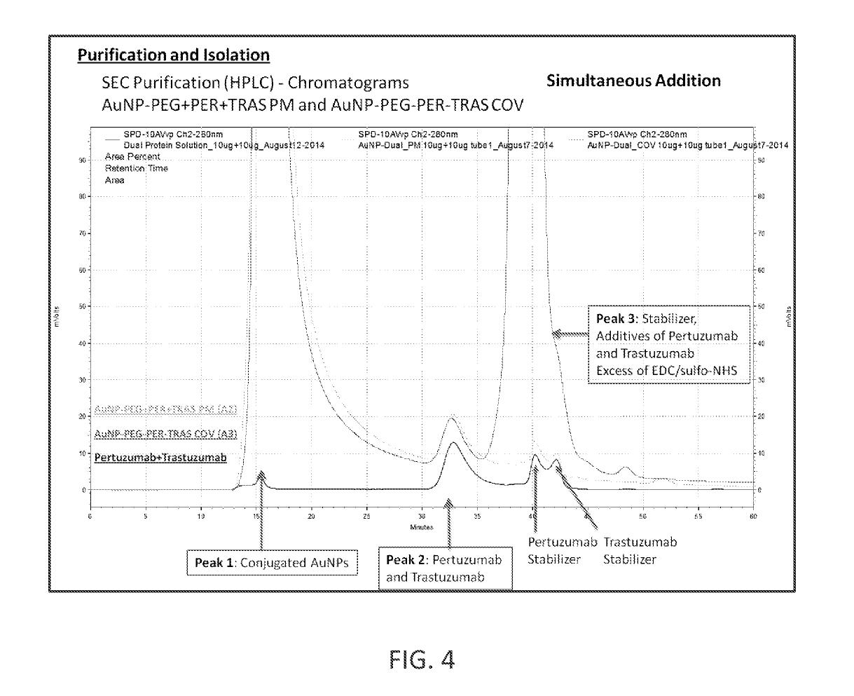

FIG. 4 are HPLC chromatograms for the two conjugates AuNP-PEG+PER+TRAS PM (A2) and AuNP-PEG-PER-TRAS COV (A3). Both A2 and A3 were prepared by simultaneous addition as illustrated in FIG. 1.

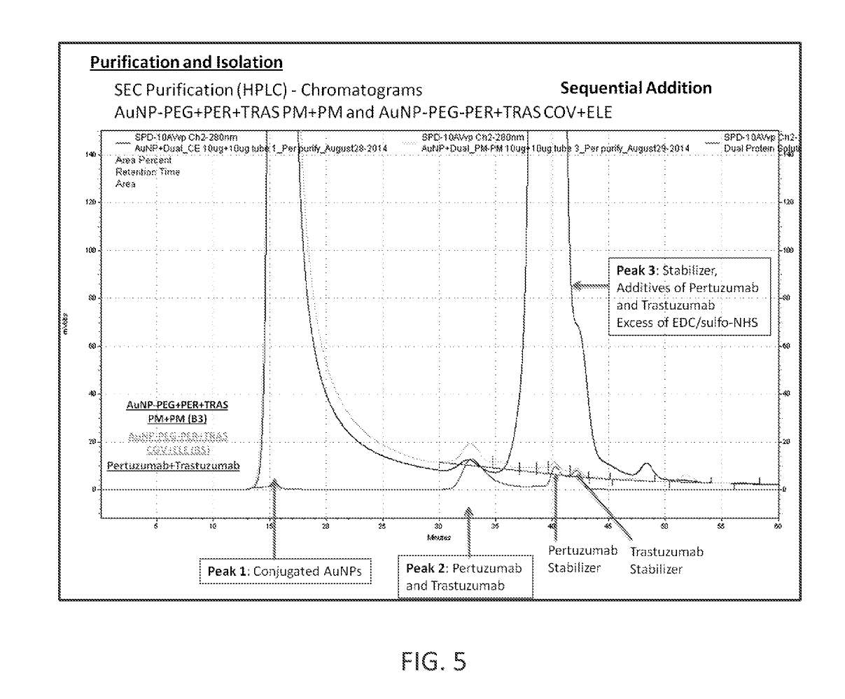

FIG. 5 includes data for dual human antibodies bonded to gold nanoparticles, HPLC chromatograms of conjugates AuNP-PEG+PER+TRAS PM+PM (B3) and AuNP-PEG-PER+TRAS COV+ELE (B5). Both B3 and B5 were prepared by sequential addition as illustrated in FIG. 2.

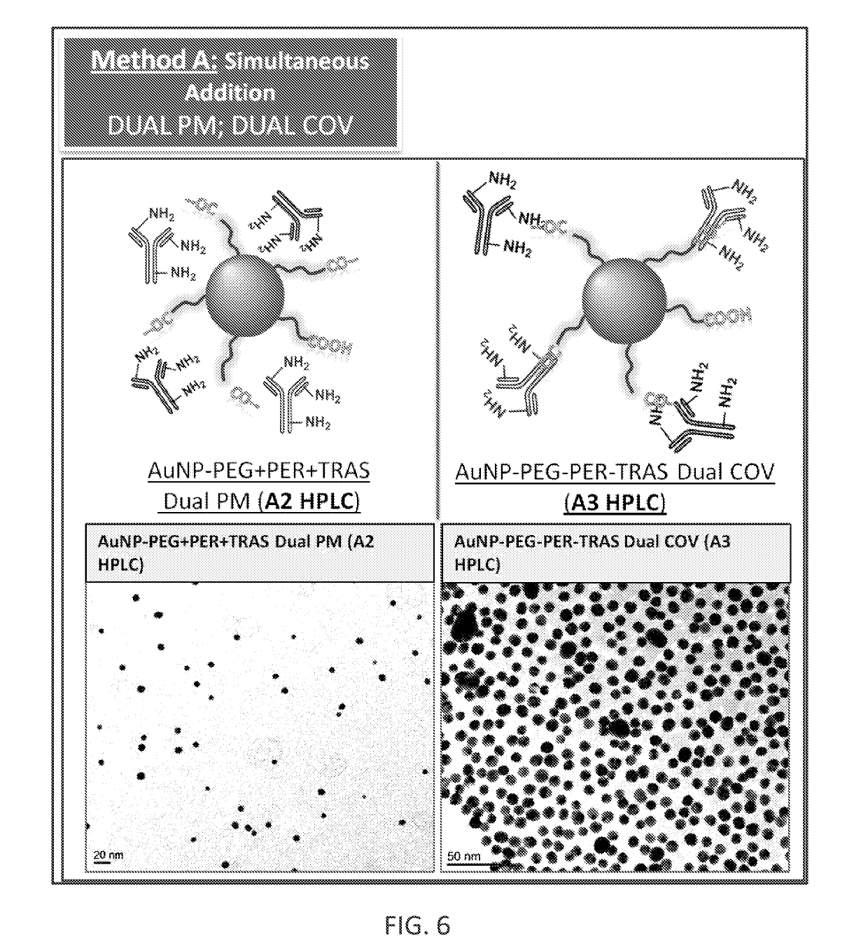

FIG. 6 includes schematic representations and TEM images of conjugates AuNP-PEG+PER+TRAS Dual PM (A2 HPLC) shown in left panel, and AuNP-PEG-PER-TRAS Dual COV (A3 HPLC) shown in right panel, both prepared by simultaneous addition (i.e., method A), with purification step for each by HPLC in accordance with FIGS. 1 and 3. HPLC″ denotes method of purification.

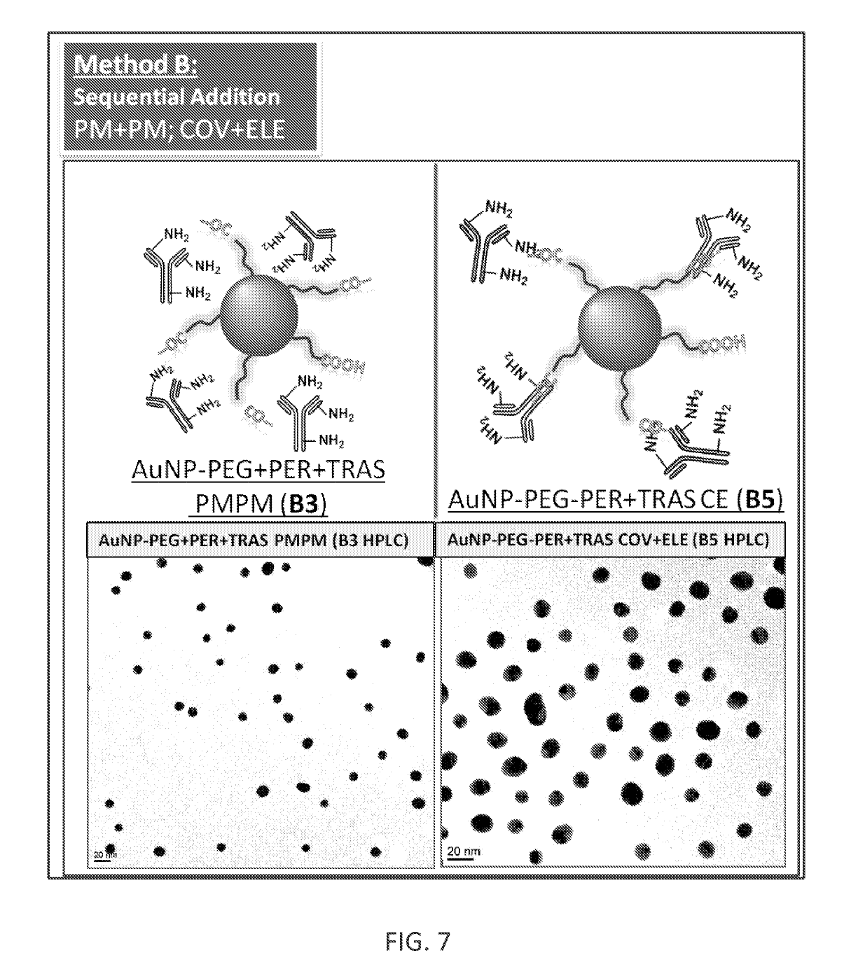

FIG. 7 includes schematic representations and TEM images of conjugates AuNP-PEG+PER+TRAS PMPM (B3 HPLC) shown in left panel, and AuNP-PEG-PER+TRAS CE (B5 HPLC) shown in right panel, both prepared by sequential addition (i.e., method B), with purification step for each by HPLC in accordance with FIGS. 2 and 3.

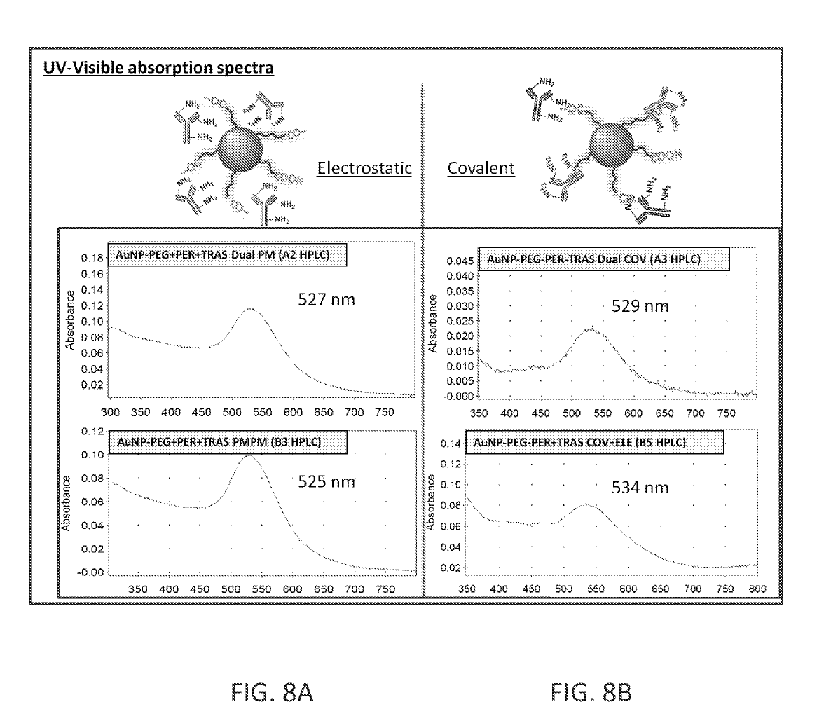

FIGS. 8A and 8B include UV-Vis spectra for dual human antibodies bond to gold nanoparticle conjugates AuNP-PEG+PER+TRAS Dual PM (A2 HPLC) and AuNP-PEG+PER+TRAS PMPM (B3 HPLC) formed by electrostatic bonding (FIG. 8A) with simultaneous and sequential methods, respectively, and for conjugates AuNP-PEG-PER-TRAS Dual COV (A3 HPLC) and AuNP-PEG-PER+TRAS CE (B5 HPLC) formed by covalent bonding (FIG. 8B) with simultaneous and sequential methods, respectively. Note, however, that while B5 is under the heading labeled “Covalent”, only the first bond formed in the sequential process (namely, PEG-PER) is covalent; whereas the second bond (namely, PER+TRAS) was formed by electrostatic method—hence, the tag COV+ELE.

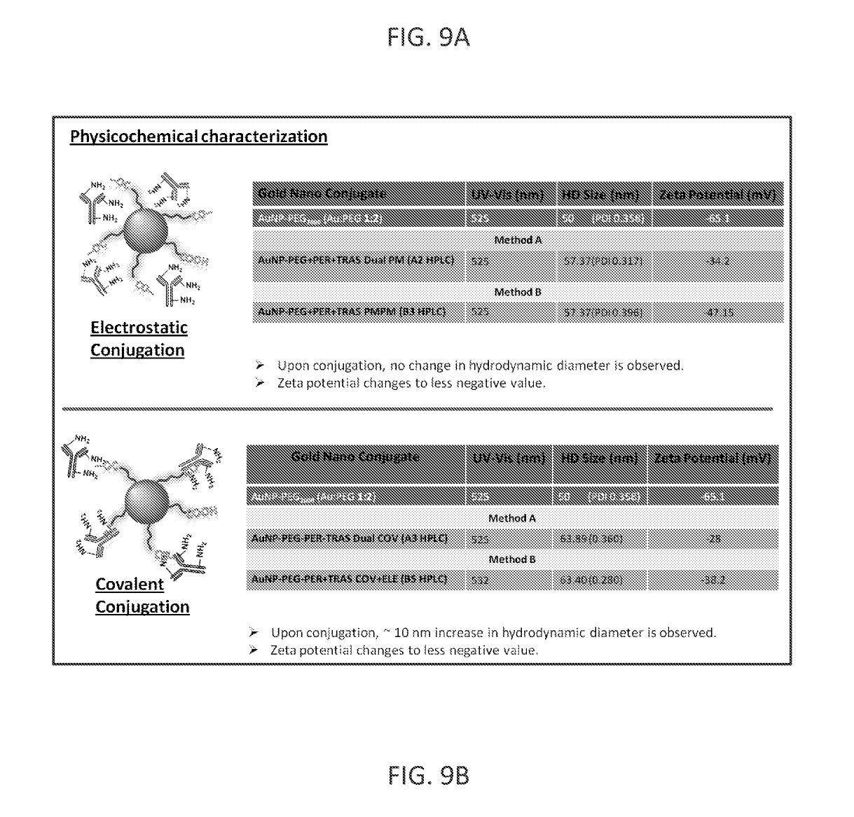

FIGS. 9A and 9B are measured physiochemical values for characterization of selected AuNP-dual antibody conjugates, including location of the plasmon absorption peak in UV-Vis spectra, hydrodynamic diameter, and zeta potential. The molecular weight of the PEG linker in a AuNP-dual antibody conjugate for the conjugates listed is 2000 kDa. As indicated by Au:PEG 1:2, the ratio of AuNP to PEG is 1:2.

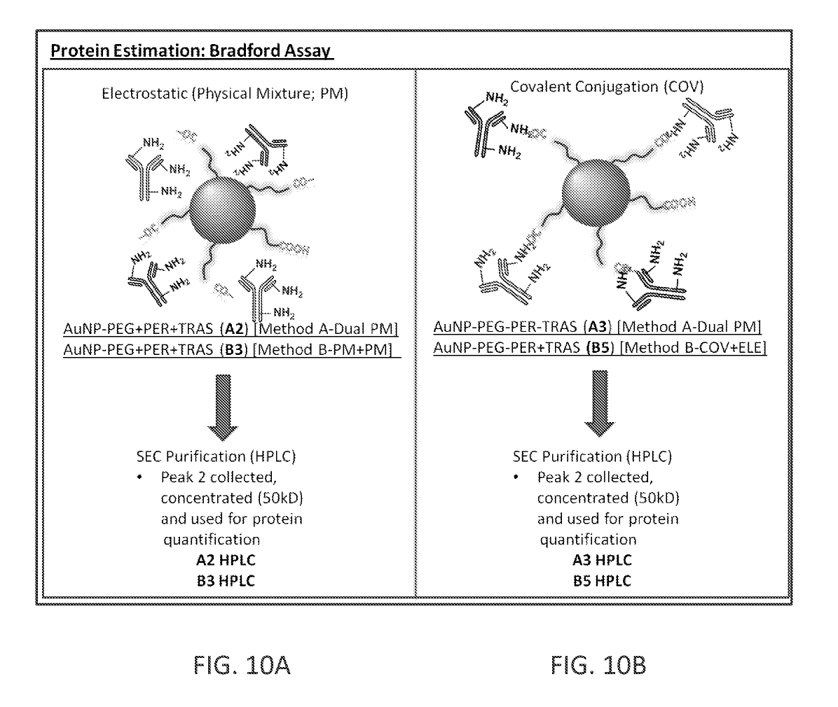

FIGS. 10A and 10B are representations for the Bradford assay processes (listed at bottom of each figure) utilized to obtain AuNP—dual antibody conjugates AuNP-PEG+PER+TRAS Dual PM (A2) formed by simultaneous method and AuNP-PEG+PER+TRAS PM+PM (B3) formed by sequential method (both shown in left panel), and for conjugates AuNP-PEG-PER-TRAS Dual COV (A3) formed by simultaneous method and AuNP-PEG-PER+TRAS COV+ELE (B5) formed by sequential method (both shown in FIG. 10B).

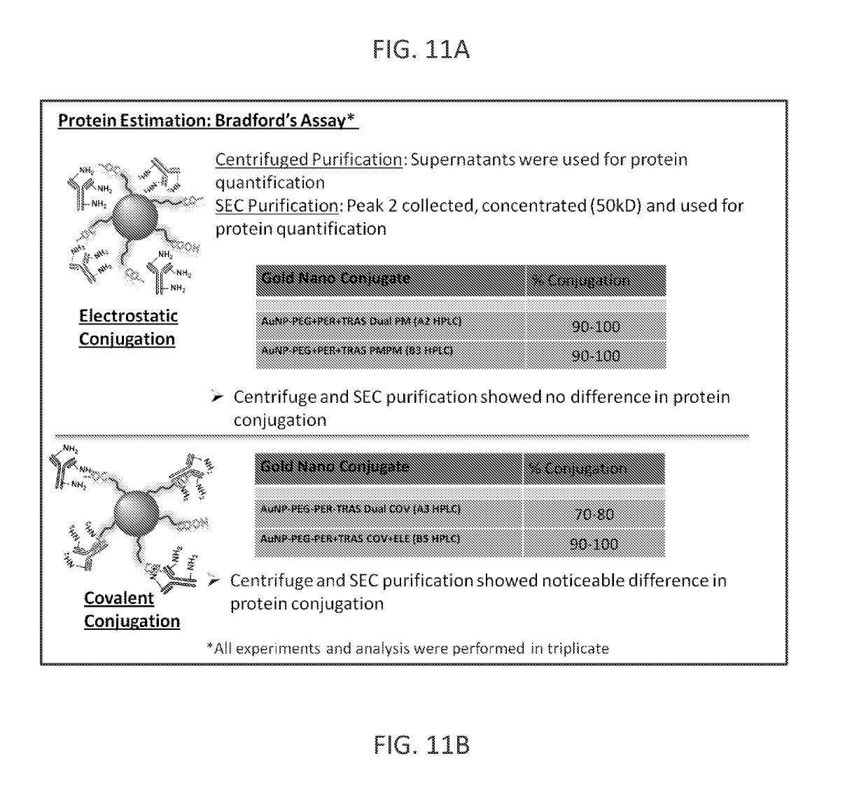

FIGS. 11A-11B include protein estimation values measured by Bradford assay for the AuNP-dual antibody conjugates A2 HPLC, B3 HPLC, A3 HPLC, and B5 HPLC, listed as % conjugation.

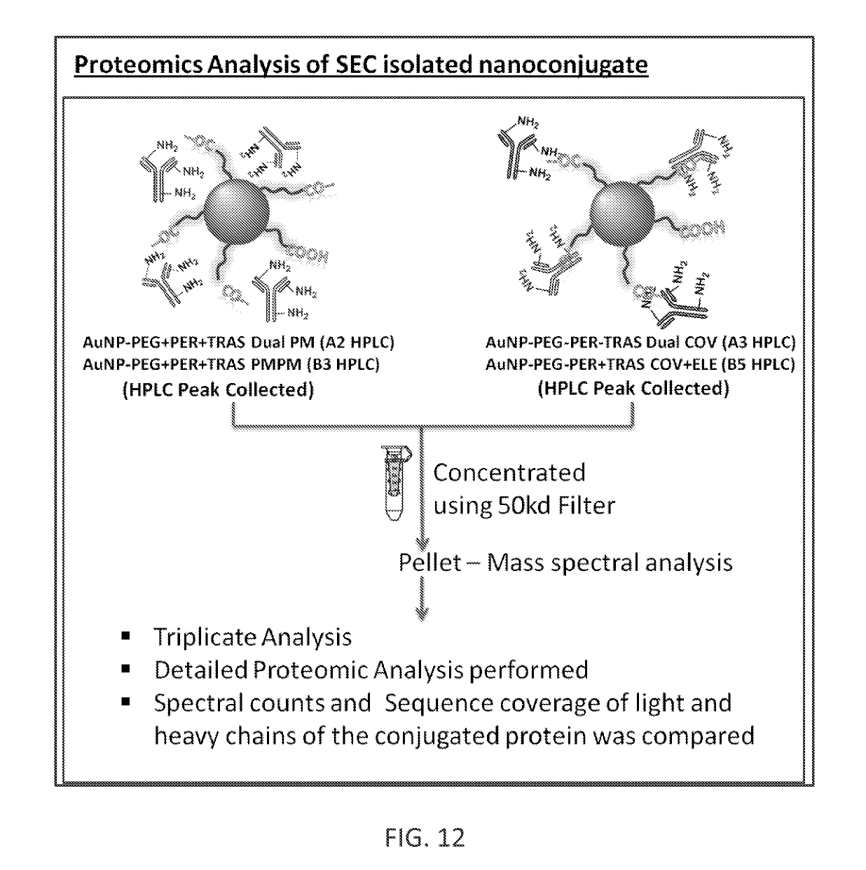

FIG. 12 shows high pressure liquid chromatography (HPLC) for purification and size exclusion chromatography (SEC) process utilized for proteomics analysis for four isolated conjugates; namely, A2 HPLC, B3 HPLC, A3 HPLC, and B5 HPLC.

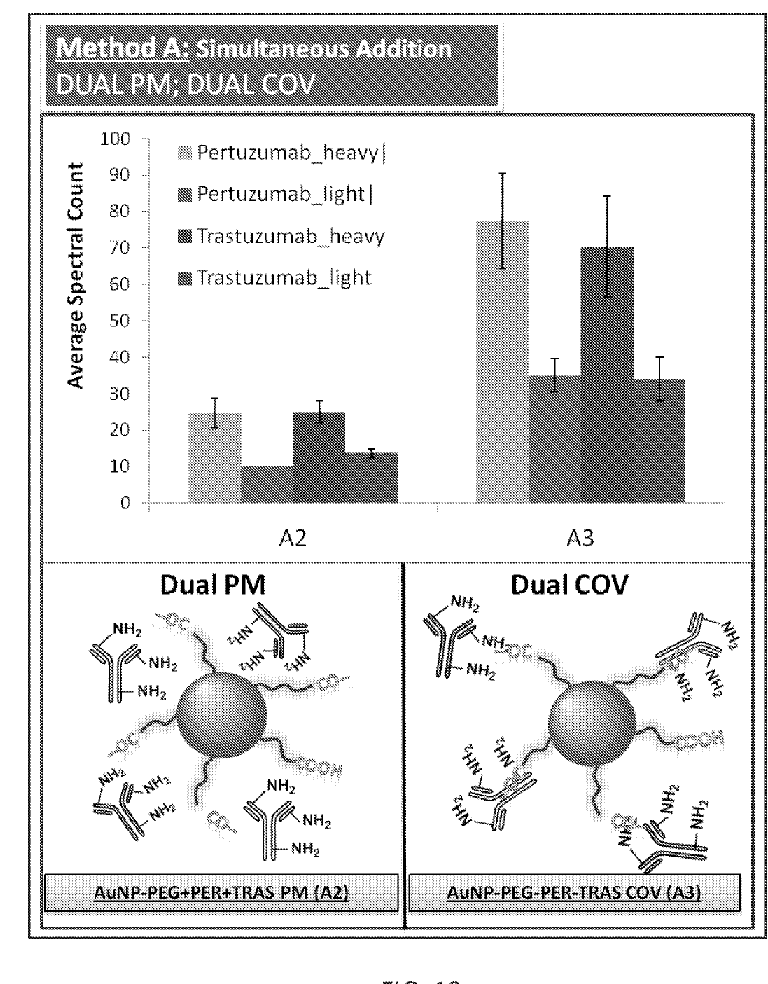

FIG. 13 includes histograms showing average spectral counts for both heavy and light chains for -dual antibody conjugates PEG+PER+TRAS Dual PM (A2) and AuNP-PEG-PER-TRAS Dual COV (A3), both of which were formed by simultaneous addition (as in FIG. 1). A schematic of each conjugate is also shown.

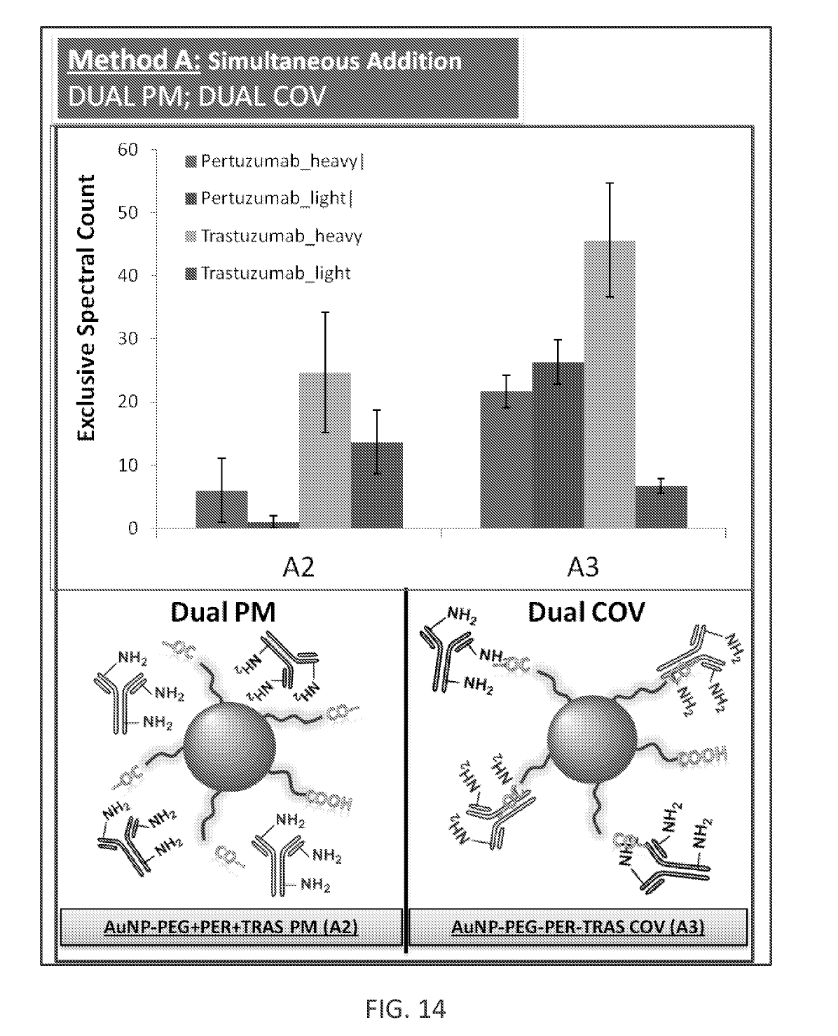

FIG. 14 includes histograms showing exclusive spectral counts for both heavy and light chains for -dual antibody conjugates PEG+PER+TRAS Dual PM (A2) and AuNP-PEG-PER-TRAS Dual COV (A3), both of which were formed by simultaneous addition (as in FIG. 1). A schematic of each conjugate is also shown.

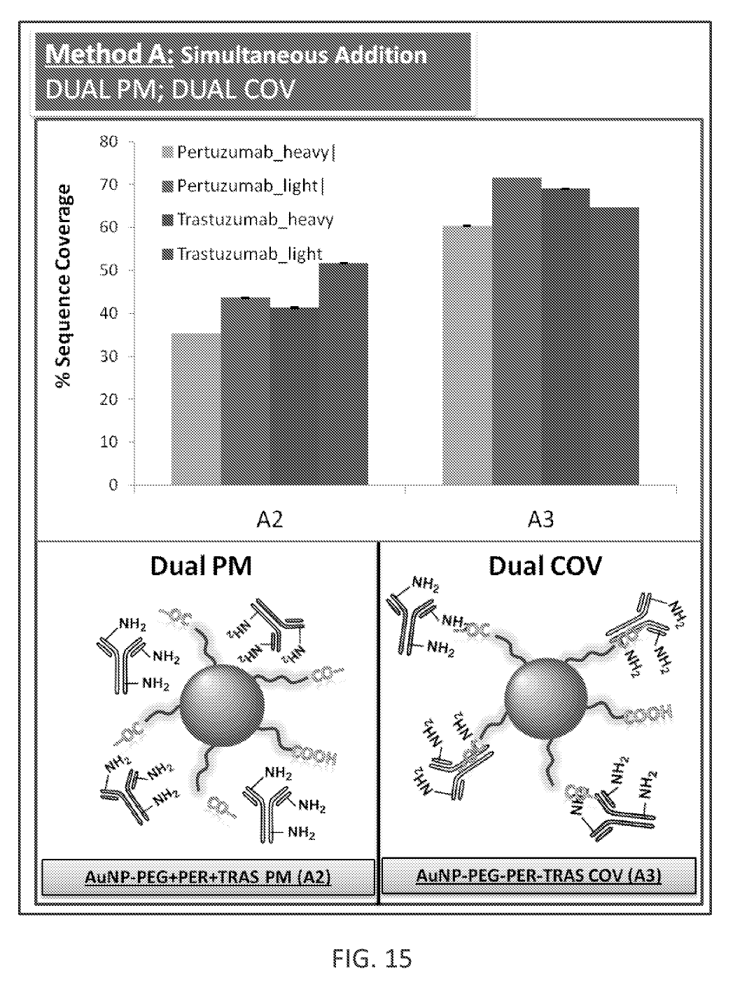

FIG. 15 includes histograms showing % sequence coverage for both heavy and light chains for -dual antibody conjugates PEG+PER+TRAS Dual PM (A2) and AuNP-PEG-PER-TRAS Dual COV (A3), both of which were formed by simultaneous addition (FIG. 1). A schematic of each conjugate is also shown.

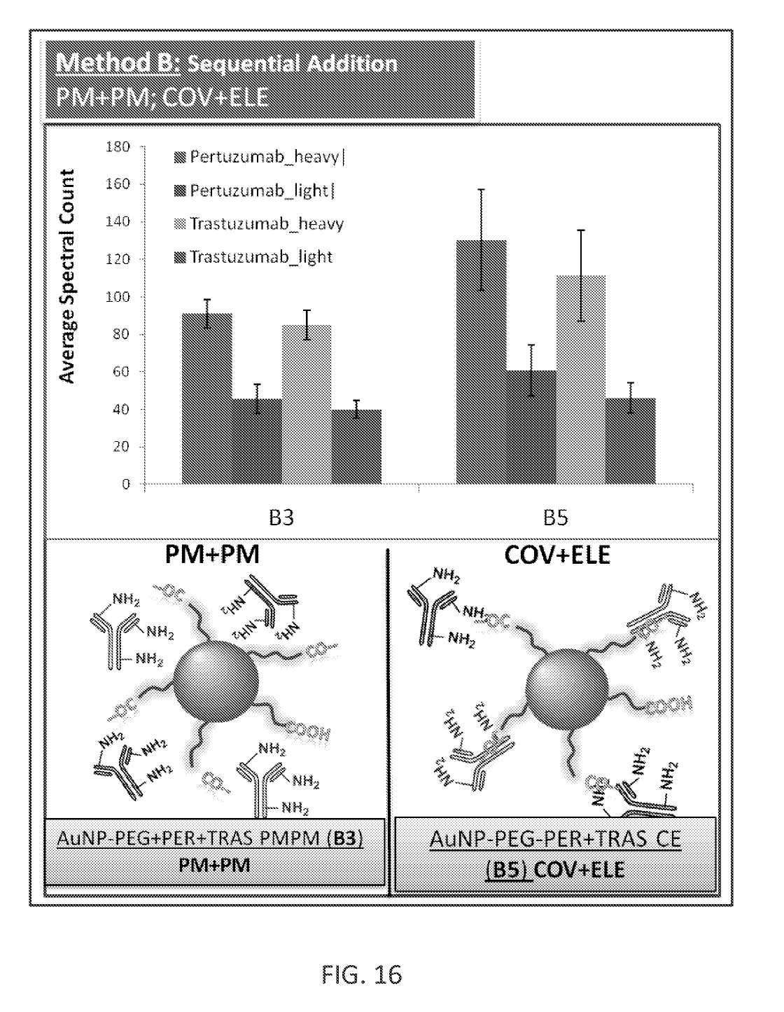

FIG. 16 includes histograms showing average spectral counts for both heavy and light chains for -dual antibody conjugates AuNP-PEG+PER+TRAS PM+PM (B3) and AuNP-PEG-PER+TRAS COV+ELE (B5), both of which were formed by sequential addition (FIG. 2). A schematic of each conjugate is also shown.

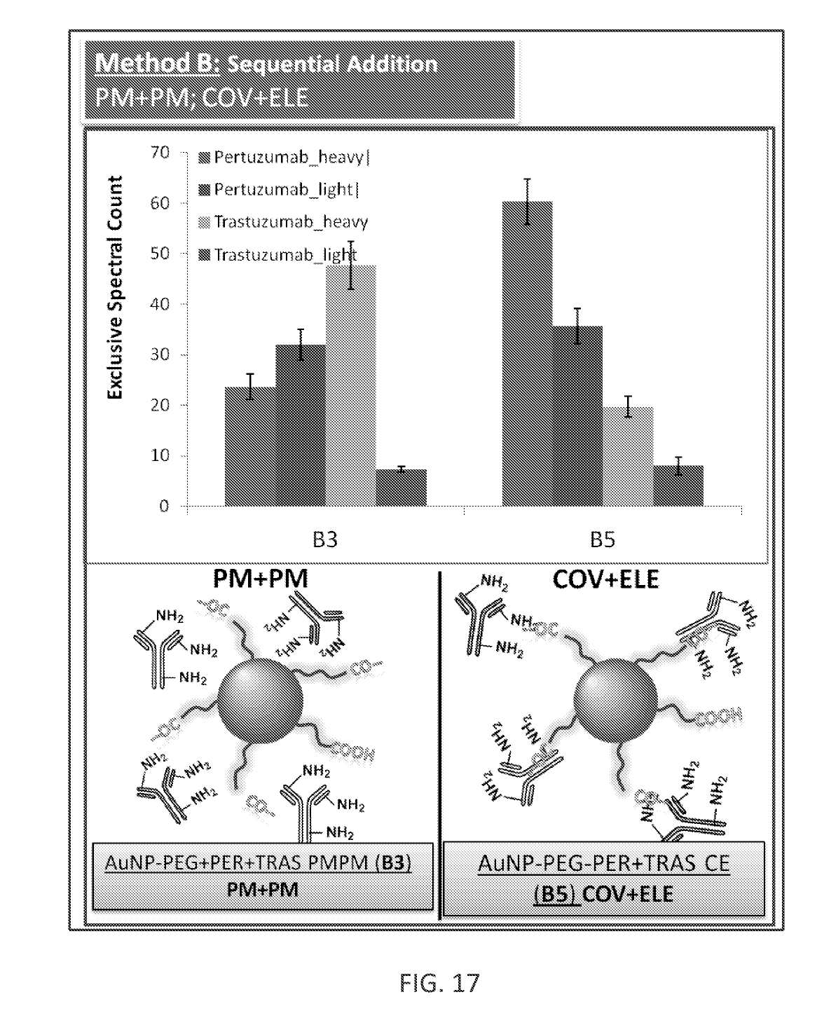

FIG. 17 includes histograms showing exclusive spectral counts for both heavy and light chains for -dual antibody conjugates AuNP-PEG+PER+TRAS PM+PM (B3) and AuNP-PEG-PER+TRAS COV+ELE (B5), both of which were formed by sequential addition (FIG. 2). A schematic of each conjugate is also shown.

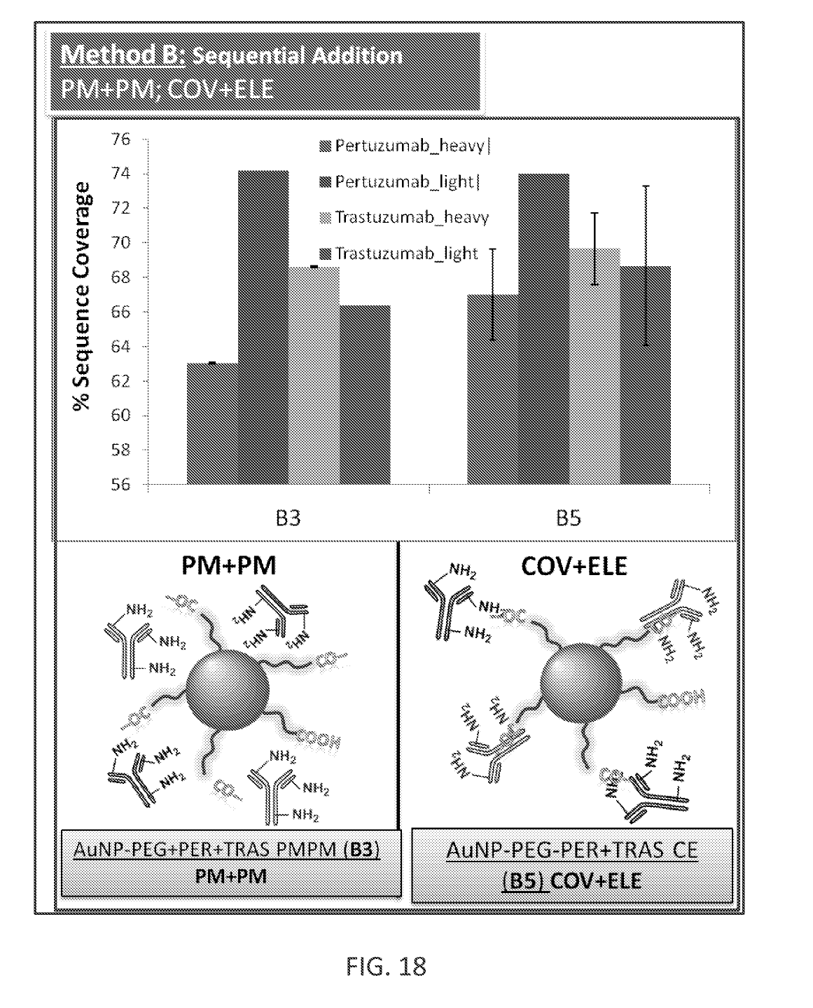

FIG. 18 includes values, shown as histograms bars, for % sequence coverage for both heavy and light chains for -dual antibody conjugates AuNP-PEG+PER+TRAS PM+PM (B3) and AuNP-PEG-PER+TRAS COV+ELE (B5), both of which were formed by sequential addition (reference FIG. 2). A schematic of each conjugate is also shown.

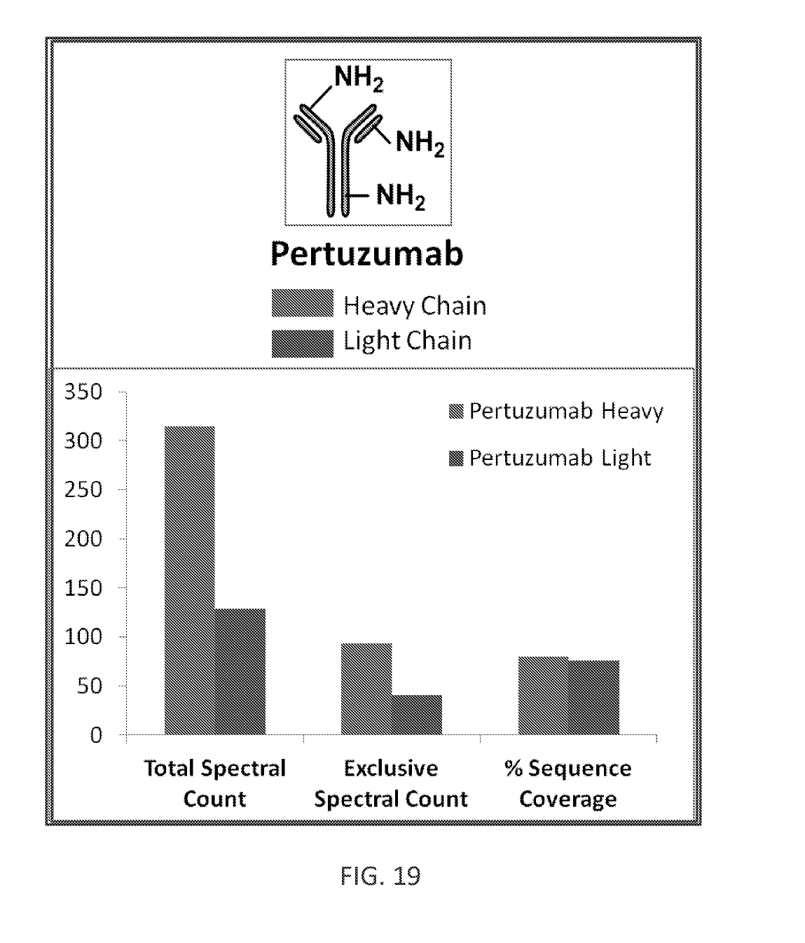

FIG. 19 includes values, shown as histogram bars, for total spectral count, exclusive spectral count, and % sequence coverage values obtained for pertuzumab heavy chain and pertuzumab light chain. The heavy and light chain values are placed side-by-side to show comparative values for heavy and light chains for total spectral count, exclusive spectral count, and % sequence coverage.

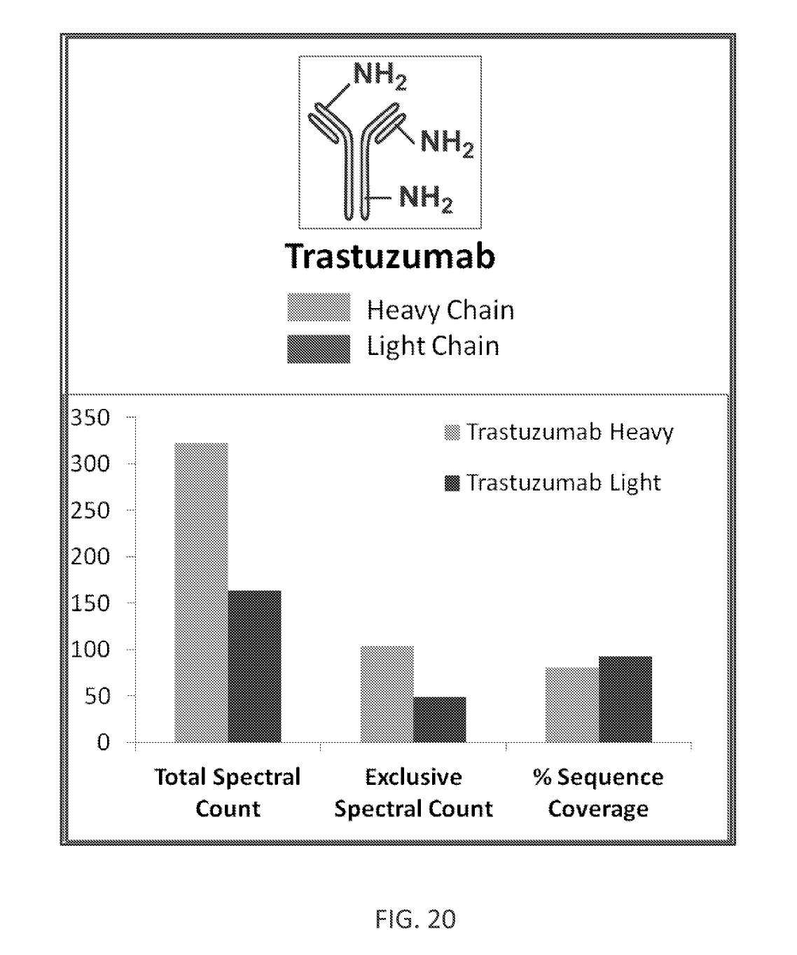

FIG. 20 includes values, shown as histogram bars, for total spectral count, exclusive spectral count, and % sequence coverage obtained for trastuzumab heavy chain and pertuzumab light chain. The heavy and light chain values are placed side-by-side to show comparative values for each of the following: heavy and light chains for total spectral count, exclusive spectral count, and % sequence coverage.

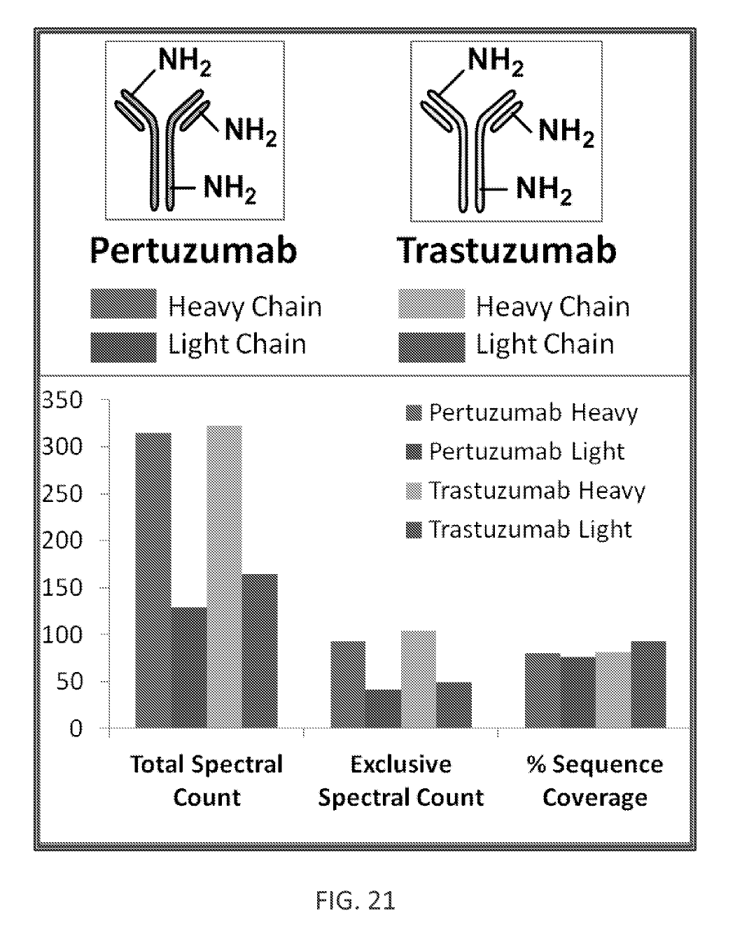

FIG. 21 includes values, shown as histogram bars, for total spectral count, exclusive spectral count, and % sequence coverage, represented as histogram bars, for each of the following: pertuzumab heavy chain, pertuzumab light chain, trastuzumab heavy chain, and trastuzumab light chain.

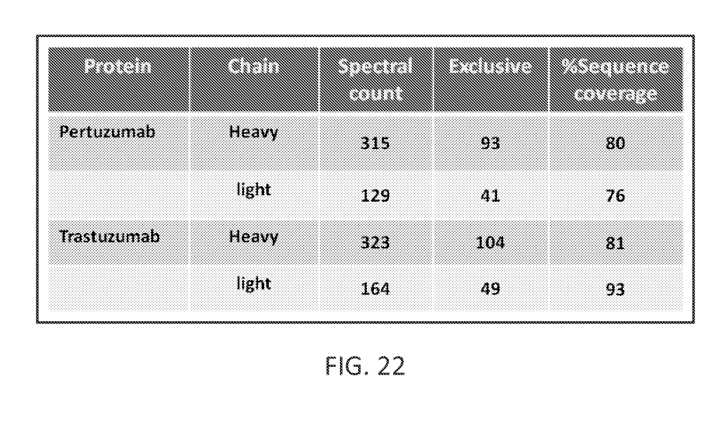

FIG. 22 includes (Total) spectral count, exclusive (spectral count) and % sequence coverage for each of the following: pertuzumab heavy chain and light chain, and for pertuzumab heavy chain and light chain.

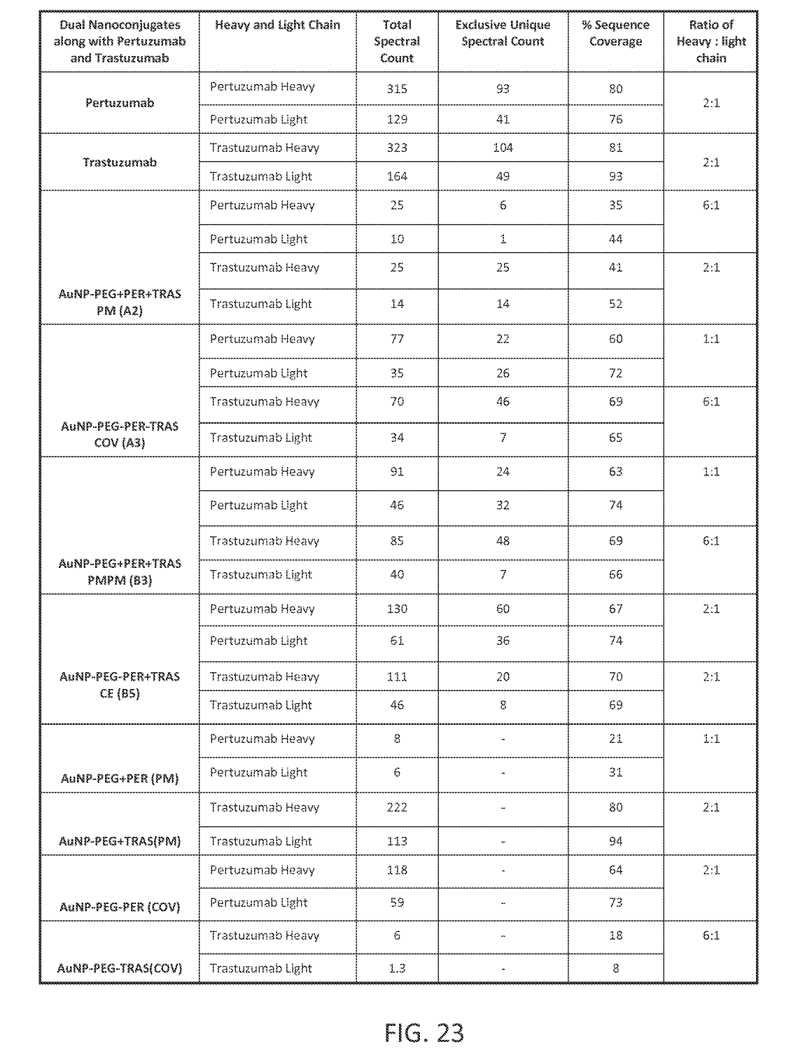

FIG. 23 includes Total spectral count, exclusive unique spectral count, % sequence coverage and ratio (heavy chain):(light chain) for each of the following: heavy chain and light chain for pertuzumab, trastuzumab, AuNP-PEG+PER+TRAS PM (A2), AuNP-PEG-PER-TRAS COV (A3), AuNP-PEG+PER+TRAS PMPM (B3), AuNP-PEG-PER+TRAS CE (B5), AuNP-PEG-PER (PM), AuNP-PEG+TRAS (PM), and AuNP-PEG-PER (COV).

Details of example experiments used to generate date discussed above will now be discussed.

In a preferred embodiment, citrate coated gold nanoparticles were made, and multiple type human antibodies, herein pertuzumab and trastuzumab, were bonded to the gold nanoparticle by two different bonding methods; namely, by electrostatic bonding and by covalent bonding. Tools based on proteomics were used to characterize the multiple type antibodies—gold nanoparticle conjugates with a PEG linker. An example would be labeled AuNP-PEG-PER-TRAS, and represents covalent bonding of both pertuzumab and trastuzumab to a AuNP (each bonded directly to the AuNP via a PEG linker).

Synthesis of AuNP-PEG-PER-TRAS conjugates: Citrate stabilized gold nanoparticles were prepared by a modified Frens and Turkevich procedure to ensure complete saturation of AuNPs to yield particles of uniform size and shape (ACS NANO 2013, 7, 1129). The citrate ions were replaced by direct chemisorption of bifunctional polyethylene glycol (PEG) linker that contained thiol and end terminal carboxyl functionality. Dual antibody conjugates were prepared by two different methods, Method A and method B. Preferred Spheres were of 15-20 nm diameter and rods of ˜50 nm length and 3:1 aspect ratio. In preferred embodiments, nanoparticles with diameter or rods with length in the range of 3-100 nm are used, and with about 35 nm being most preferred.

In method A, dual conjugation was achieved by simultaneous addition of two antibodies to AuNP-PEG conjugate to form dual antibody conjugates. These dual antibody conjugates were prepared by utilizing both electrostatic and covalent conjugation techniques. For electrostatic attachment, the conjugation was performed by mixing of two antibodies (10 μg of each antibody) AuNP-(PEG-2000)-COOH (1) without activation of carboxyl groups. For covalent conjugation, the terminal carboxyl groups of AuNP-(PEG-2000)-COOH (1) were activated using 1-Ethyl-3-[3-dimethylaminopropyl] carbodiimide hydrochloride/N-hydroxysulfosuccinimide ((EDC)/(Sulfo NHS)) procedure in 4-morpholinoethanesulfonic acid (MES) buffer (pH=4.5) followed by addition of both antibodies (20 μg) simultaneously. The dual antibody conjugates obtained by both electrostatic (A2) and covalent (A3) attachments were purified by HPLC and isolated by SEC processes.

In method B, dual conjugation was achieved by sequential addition of antibodies. As a first step, pertuzumab (PER) was attached to a citrate coated gold nanoparticle with a polyethylene glycol linker (PEG). The resulting conjugate (AuNP-PEG-PER) was prepared by two alternative methods, as selected, with the two methods being either by covalent or electrostatic attachment. The AuNP-PEG-PER was purified by HPLC and isolated by SEC procedures, followed by addition of a second antibody, trastuzumab. In electrostatic attachment, the pertuzumab was added to (1) by simple mixing (physical mixing (PM)) in absence activating agents, purified by HPLC, and isolated by SEC, and further incubated with trastuzumab. The physiadsorbed dual antibody gold conjugate was finally purified by HPLC and isolated by SEC (B3). In covalent-electrostatic attachment, AuNP-PEG-PER was first prepared by activation of AuNP-(PEG-2000)-COOH (1) with EDC/Sulfo NHS followed by addition of 10 μg of pertuzumab and the crude mixture was passed through an SEC column. The SEC isolated conjugate was concentrated to 200 μl and trastuzumab (10 μg) was added, incubated, and finally isolated by SEC procedure to obtain the covalently and electrostatically attached dual antibody conjugate (B5).General Results for Sequential Addition of Antibodies:

B3—Spectral counts of both proteins are similar suggesting their equal abundance on the gold nanoparticle.

B5—Slight variation is observed in spectral counts between the two proteins suggesting that these are conjugated and the abundance is similar.

Sequential addition resulted in equal protein abundance on AuNPs compared to simultaneous addition. The experimental nanoconjugates were formed by two different bonding processes; namely, by electrostatic conjugation and by covalent conjugation.

For electrostatic attachment, with the conjugation formed simultaneously, the two antibodies were mixed and added in quantities in a 1:1 mass ration of 10 μg each to AuNP-(PEG-2000)-COOH without activation of the carboxyl groups.

In covalent conjugation, with the conjugation formed simultaneously, the terminal carboxyl group of AuNP-(PEG-2000)-COOH was activated using a 1-Ethyl-3-[3-dimethylaminopropyl] carbodiimide hydrochloride/N-hydroxysulfosuccinimide ((EDC)/(Sulfo NHS)) procedure in 4-morpholinoethanesulfonic acid (MES) buffer (pH=4.5) followed by addition of both antibodies, herein, pertuzumab and trastuzumab, labeled as PER and TRAS, respectively, simultaneously. (Note: total mass added was 20 μg).

The dual antibody nanoconjugates obtained either by electrostatic bonding or by covalent bonding were then purified by HPLC or isolated by SEC technique.

Method B: In method B, dual conjugation was achieved by sequential addition of antibodies. As a first step, for binding PER and TRAS, AuNP-PEG-PER conjugate was prepared by bonding PER to AuNP-(PEG-2000) by either covalent attachment or by electrostatic attachment, then purified by HPLC and isolated by SEC procedure, and then followed by addition of the second antibody TRAS.

In electrostatic attachment, PER was added to AuNP-PEG by simple, physical mixing (PM) in the absence of any activating agents, purified by HPLC and isolated by SEC, and then further incubated with TRAS. The physioadsorbed dual antibody-AuNP conjugate was finally purified by HPLC and isolated by SEC, labeled as AuNP-PER+PER+TRAS PMPM.

In covalent-electrostatic attachment, AuNP-PEG-PER conjugate was first prepared by activation of AuNP-(PEG-2000)-COOH with EDC/sulfo NHS, followed by addition of 10 μg of pertuzumab. The mixture was then passed through a SEC column. The SEC isolated conjugate was concentrated to 200 μL and 10 μg of trastuzumab was added, incubated, and finally isolated by SEC procedure to obtain the covalently and electrostatically attached dual antibody nanoconjugate, labeled as AuNP-PEG-PER-TRAS CE. The term “CE” represents covalent followed by electrostatic. An alternate terminology used herein for this two-step process is “COV ELE.”

The following definitions and terms are provided to aid reading of experimental data.

Dual COV: Simultaneous addition of both antibodies (PER and TRAS) with to AuNP-PEG-COOH following activation of COOH terminal groups, forming to AuNP-PEG-PER-TRAS Dual COV.

Dual PM: Simultaneous addition of both antibodies to AuNP-PEG-COOH without activation of COOH terminal groups.

PMPM: Sequential addition of both antibodies (PER and TRAS) to AuNP-PEG-COOH without activation of COOH terminal group, with a purification step between additions (purification by high pressure liquid chromatography (HPLC) and isolation of AuNP-PEG+PER) by size exclusion chromatography (SEC), before adding TRAS), forming AuNP-PEG+PER+TRAS PMPM

COV ELE: Sequential addition to AuNP-PEG-COOH, with first antibody (PER) covalently bonded following activation of COOH terminal groups that produces covalent bonding, then purification, followed by addition of second antibody (TRAS) to AuNP-PEG-PER using physical mixing (i.e., without activation of COOH terminal groups) that leads to electrostatic bonding, forming AuNP-PEG+TRAS COV ELE.

Let Ab1 represent a human antibody, such as pertuzumab.

Let Ab2 represent a human antibody, such as trastuzumab.

1. Preparation of dual covalently bonded human antibody—AuNP conjugates of the invention that is formed by simultaneous mixing, e.g., AuNP-PEG-Ab1-Ab2 Dual COV, including the following steps:

Preparation of citrate coated AuNPs in aqueous solution;

Preparation of AuNP-PEG-COOH in solution;

Activation of carboxyl terminated AuNP-PEG-COOH using EDC/Sulfo-NHS;

Simultaneous addition of Ab1 and Ab2 antibodies to AuNP-PEG with activated terminal groups solution; and

Purification by HPLC, and isolation by SEC.

2. Preparation of dual electrostatically bonded human antibody—AuNP conjugates of the invention that is formed by simultaneous mixing, e.g., AuNP-PEG+Ab1+Ab2 Dual PM, included the steps:

Preparation of citrate coated AuNPs in aqueous solution;

Preparation of AuNP-PEG-COOH in solution;

Simultaneously adding Ab1 and Ab2 antibodies to AuNP-PEG-COOH solution;

and

Purification by HPLC, and isolation by SEC.

3. Preparation of dual electrostatically bonded human antibody—AuNP conjugates of the invention that is formed by sequential mixing, e.g., AuNP-PEG+Ab1+Ab2 PM+PM, including the steps:

Preparation of citrate coated AuNPs in aqueous solution;

Preparation of AuNP-PEG-COOH in solution;

Addition of Ab1 antibody to solution containing AuNP-PEG-COOH;

Purification of solution by HPLC, and isolation by SEC

Addition of Ab2 antibody to isolated AuNP-PEG+Ab1 solution; and

Purification by HPLC, and isolation by SEC.

4. Preparation of covalent bonded and electrostatically bonded human antibody—AuNP conjugates of the invention that is formed by sequential mixing, e.g., AuNP-PEG-Ab1+Ab2 COV ELE, including the steps:

Preparation of citrate coated AuNPs in aqueous solution;

Preparation of AuNP-PEG-COOH in solution;

Activation of carboxyl terminated AuNP-PEG-COOH using EDC/Sulfo-NHS;

Addition of Ab1 antibody to solution containing AuNP-PEG with activated terminal groups solution;

Purification by HPLC, and isolation by SEC;

Addition of Ab2 antibody to isolated AuNP-PEG+Ab1 solution; and

Purification by HPLC, and isolation by SEC.;

Additional information pertaining to synthesis—an example

Further details for synthesis of dual antibody types bound to one gold nanoparticle are presented below in the form of examples of synthesis of a human antibody-AuNP conjugate. The first example describes synthesis of pertuzumab antibody—AuNP conjugate with covalent binding method; namely, AuNP-PEG-PER COV. The second example describes synthesis of pertuzumab antibody—AuNP conjugate with electrostatic binding method; namely, AuNP-PEG+PER PM. As such, the examples describe the first portion of the synthesis leading to a dual human antibody-AuNP conjugate wherein the first addition would be pertuzumab by either covalent binding method, or electrostatic binding method. The purpose is to provide further details on chemicals and procedures that are employed in the invention.

The above paragraph describes synthesis of dual antibody types bound to one gold nanoparticle. The same procedures can be utilized to synthesize multiple antibody type bound to one gold nanoparticle, either sequentially as described or simultaneously as described.

### Examples—Detailed Synthesis of Covalent and Electrostatic Conjugated Pertuzumab to AuNP-PEG2 (Au:PEG1:2)

Gold nanoparticles (AuNPs) were pegylated with end terminal carboxyl groups at stoichiometric ratios of Au-NP-PEG-COOH 1:2 and 1:5. Au:PEG1:2 stoichiometric nanoparticles will be hence forward be referred to as AuNP-PEG2, and Au:PEG1:5 as AuNP-PEGS. PEG is polyethylene glycol.

Activation of Carboxyl groups: Carboxyl groups in the AuNP-PEG2 were activated using 1-Ethyl-3-[3-dimethylaminopropyl] carbodiimide hydrochloride/N-hydroxysulfosuccinimide (EDC/NHS) chemistry using 4-morpholinoethanesulfonic acid (MES) buffer at pH 4.5. The EDC 10 mg and sulfo-NHS 10 mg were dissolved in 40 μl MES buffer, and then mixed together. This solution was added to the 1 ml of AuNP-PEG2 and incubated at 37° C. for 3 hrs with continuous shaking at 650 rpm. The activated AuNPs were centrifuged at 1000 revolutions per minute (rpm) for 10 min at room temperature (RT, i.e., 37° C. and excess of EDC/NHS was separated from the activated nanoparticles.

Antibody Conjugation: Pertuzumab 20 μg was added to the 200 μl of 1×PBS at pH 7.4. To this solution, the activated AuNP-PEG2 was added slowly in a drop-wise fashion. This reaction mixture was allowed to incubate at room temperature overnight with continuous shaking at 650 rpm. All reactions were performed in triplicates.

Purification by HPLC and Separation by Size Exclusion Chromatography (SEC): After overnight incubation, the reaction mixture was purified by subjecting a 200 μl sample to size exclusion chromatography. The peaks were collected as PEAK 1: AuNP-Pertuzumab at 16 min (1.5 ml peak collected volume); PEAK 2: Free pertuzumab at 30 min (1.5 ml peak collected volume); PEAK 3: EDC/NHS, histidine and other antibody additives after 40 mins (2 ml peak collected volume).

Purification by Centrifugation: The reaction mixtures were centrifuged at 15,000 rpm for 20 min at 4° C. The supernatant was collected as Sup#1. The pellet was further washed two times by 1×PBS and Sup#2 and Sup#3 were collected.

Protein quantification using Bradford Assay: Size Exclusion Chromatography (SEC) peak 2 was collected as 1.5 ml. This 1.5 ml solution was concentrated to 200 μl by 50 kD filters and used for protein analysis. 1.5 ml was also used directly for protein analysis.

Results: All supernatants were also analyzed for detecting the amount of protein unconjugated to the nanoparticles. A standard curve was developed using serial dilutions of pertuzumab and unknown samples were analyzed. Based on the results obtained, it was found that approximately 60-70% of protein was conjugated to the AuNPs by covalent conjugation.

Electrostatic conjugation: The AuNP-PEG2 was incubated with pertuzumab 20 μg without using EDC/NHS. Rest of the procedure used for conjugation was exactly same as shown above. The reaction mixture was further purified by HPLC and isolated by SEC and centrifugation and protein quantification was performed by Bradford assay.

Results: In case of physioadsorption, approximately 50-60% protein was conjugated on the surface of AuNPs.

Trastuzumab—AuNP Conjugate Synthesis

Trastuzumab—AuNP conjugate synthesis was carried out using the same steps as provided for pertuzumab—AuNP conjugate synthesis, as described above. Results and outcomes for each step were similar.

Preparation of dual covalently bonded human antibody—AuNP conjugates of the invention that is formed by simultaneous mixing, e.g., AuNP-PEG-Ab1-Ab2 Dual COV, comprising the following steps, where Ab1 represents pertuzumab and Ab2 represents trastuzumab:

Preparation of citrate coated AuNPs in aqueous solution;

Preparation of AuNP-PEG-COOH in solution;

Activation of carboxyl terminated AuNP-PEG-COOH using EDC/Sulfo-NHS;

Simultaneously addition of Ab1 and Ab2 antibodies to AuNP-PEG with activated terminal groups solution; and

Purification by HPLC, and isolation by SEC.

Preparation of dual electrostatically bonded human antibody—AuNP conjugates of the invention that is formed by simultaneous mixing, e.g., AuNP-PEG+Ab1+Ab2 Dual PM, including the steps:

Preparation of citrate coated AuNPs in aqueous solution;

Preparation of AuNP-PEG-COOH in solution;

Simultaneously adding Ab1 and Ab2 antibodies to AuNP-PEG-COOH solution; and

Purification by HPLC, and isolation by SEC.

Preparation of dual electrostatically bonded human antibody—AuNP conjugates of the invention that is formed by sequential mixing, e.g., AuNP-PEG+Ab1+Ab2 PM+PM, including the steps:

Preparation of citrate coated AuNPs in aqueous solution;

Preparation of AuNP-PEG-COOH in solution;

Addition of Ab1 antibody to solution containing AuNP-PEG-COOH;

Purification of solution by HPLC, isolation by SEC;

Addition of Ab2 antibody to isolated AuNP-PEG+Ab1 solution, and

Purification by HPLC.

Preparation of covalent bonded and electrostatically bonded human antibody—AuNP conjugates of the invention that is formed by sequential mixing, e.g., AuNP-PEG-Ab1+Ab2 COV ELE, comprising the steps:

Preparation of citrate coated AuNPs in aqueous solution;

Preparation of AuNP-PEG-COOH in solution;

Activation of carboxyl terminated AuNP-PEG-COOH using EDC/Sulfo-NHS;

Addition of Ab1 antibody to solution containing AuNP-PEG with activated terminal groups solution;

Purification by HPLC, and isolation by SEC

Addition of Ab2 antibody to isolated AuNP-PEG+Ab1 solution; and

Purification by HPLC, and isolation by SEC.

Synthesis of single human antibody pertuzumab bound to AuNP

Preparation of covalent bonded human antibody pertuzumab—AuNP conjugate, including the steps:

Preparation of citrate coated AuNPs in aqueous solution;

Preparation of AuNP-PEG-COOH in solution;

Activation of carboxyl terminated AuNP-PEG-COOH using EDC/Sulfo-NHS;

Addition of pertuzumab human antibody to solution containing AuNP-PEG with activated terminal groups solution;

Purification by HPLC, and isolation by SEC;

Preparation of covalent bonded human antibody trastuzumab—AuNP conjugate, comprising the steps:

Preparation of citrate coated AuNPs in aqueous solution;

Preparation of AuNP-PEG-COOH in solution;

Activation of carboxyl terminated AuNP-PEG-COOH using EDC/Sulfo-NHS;

Addition of trastuzumab human antibody to solution containing AuNP-PEG with activated terminal groups solution;

Purification by HPLC, and isolation by SEC.

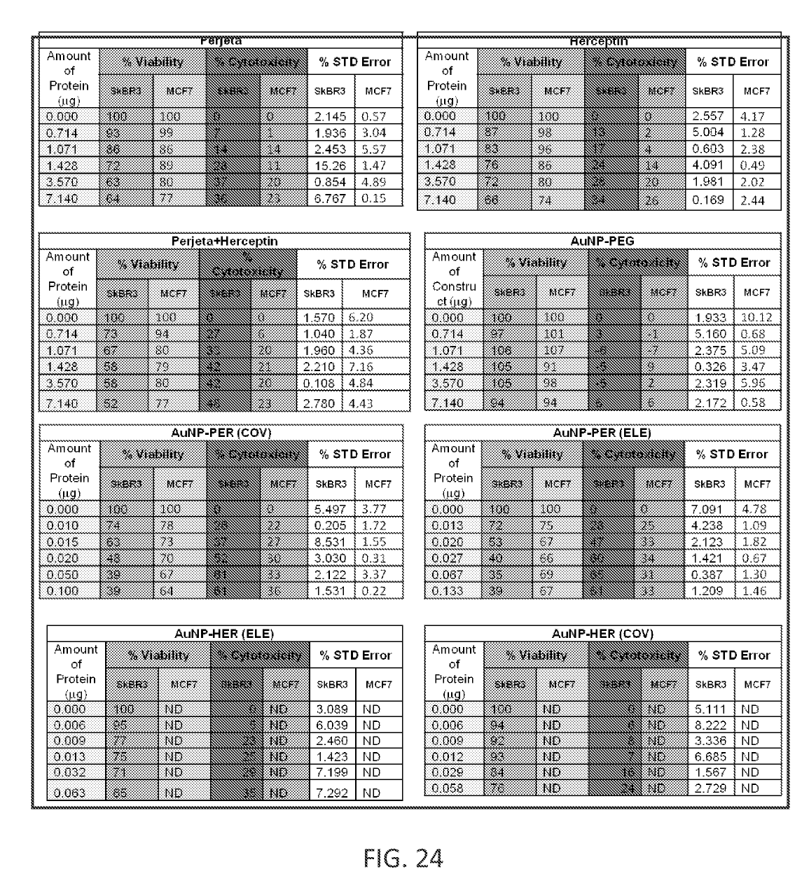

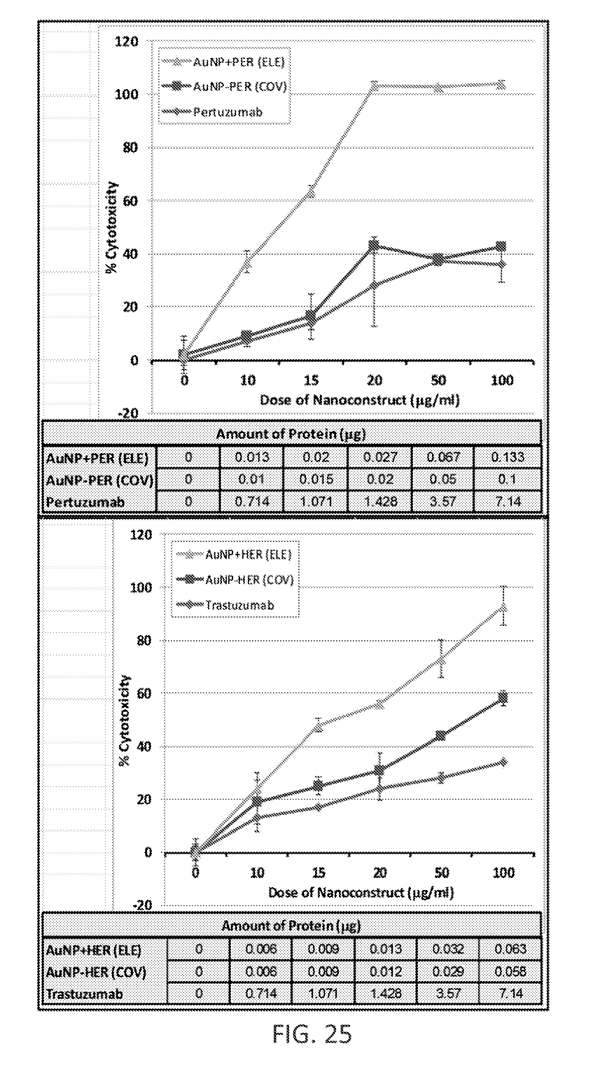

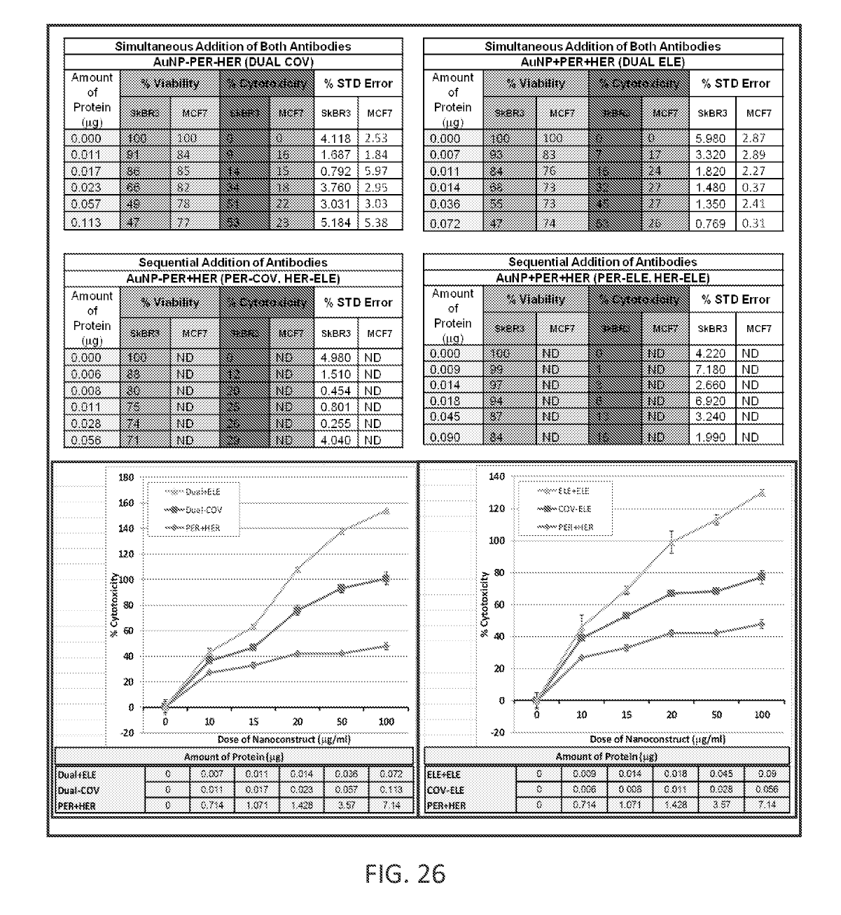

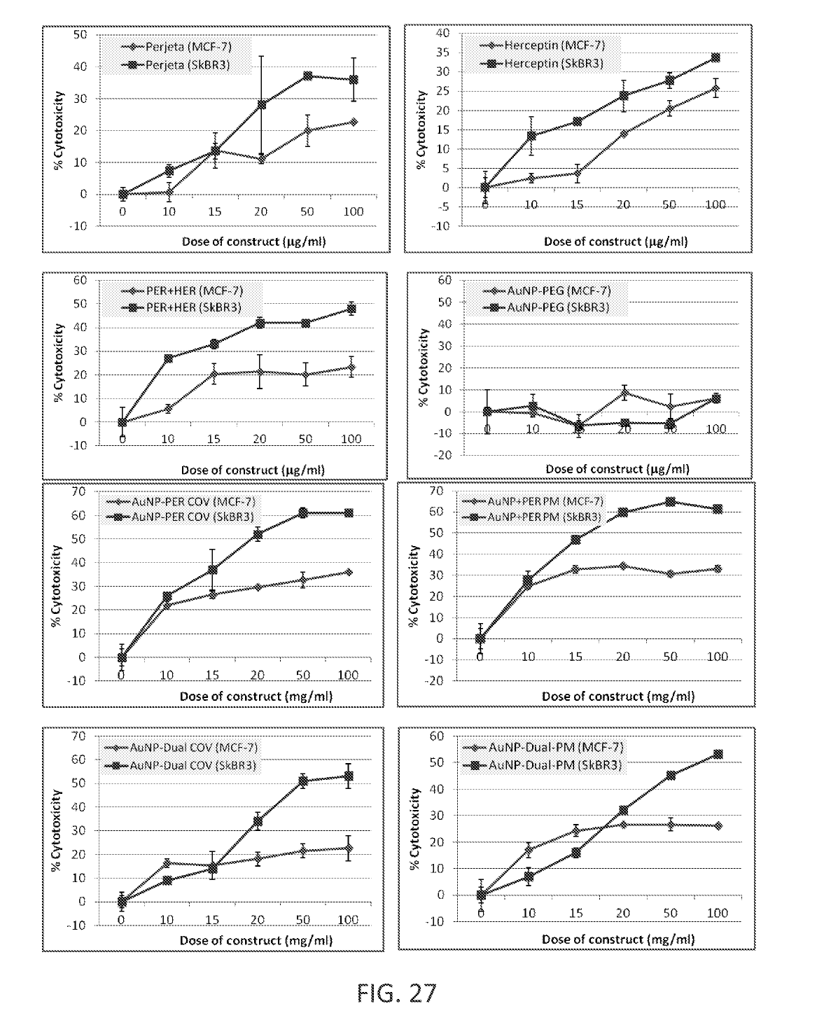

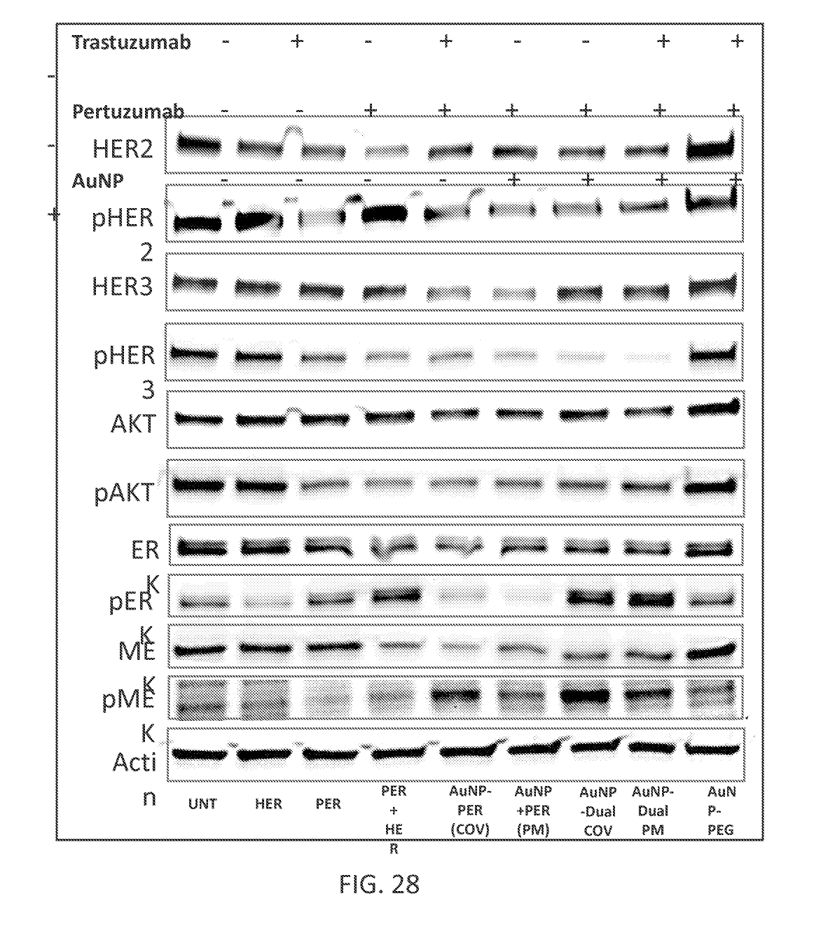

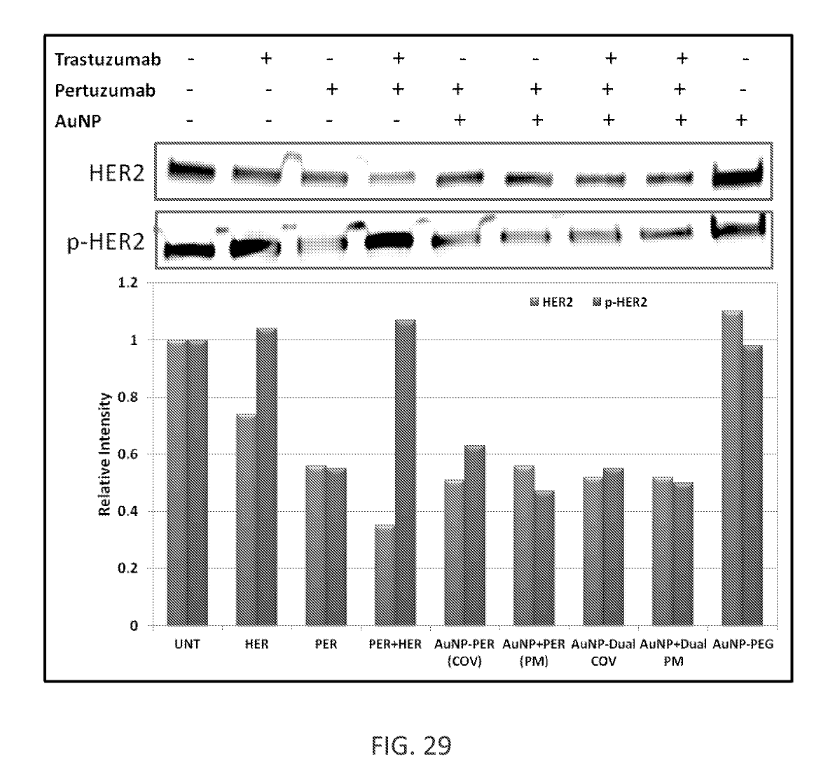

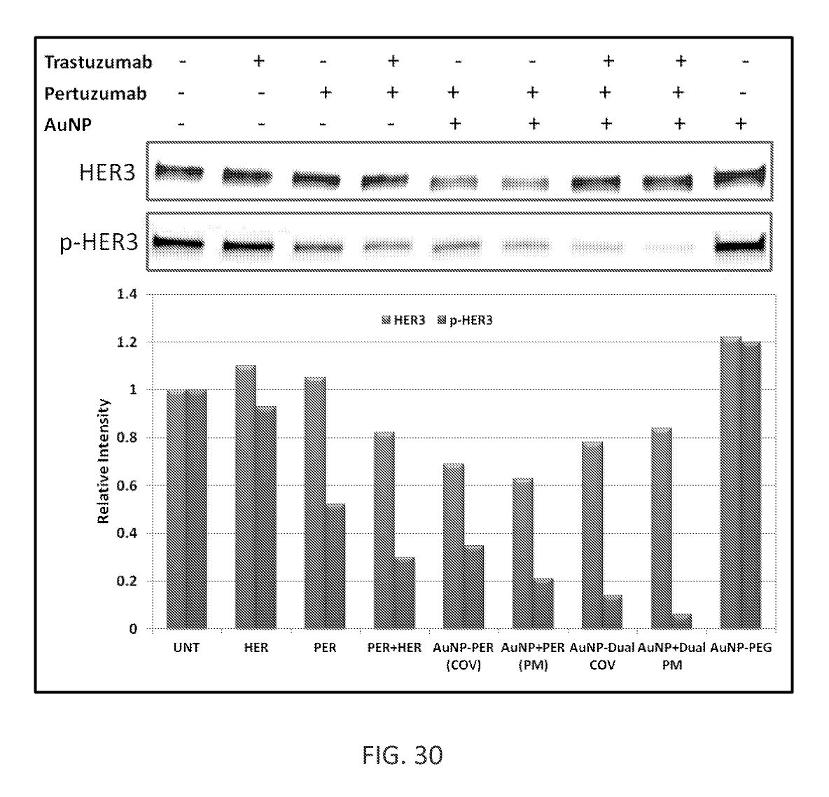

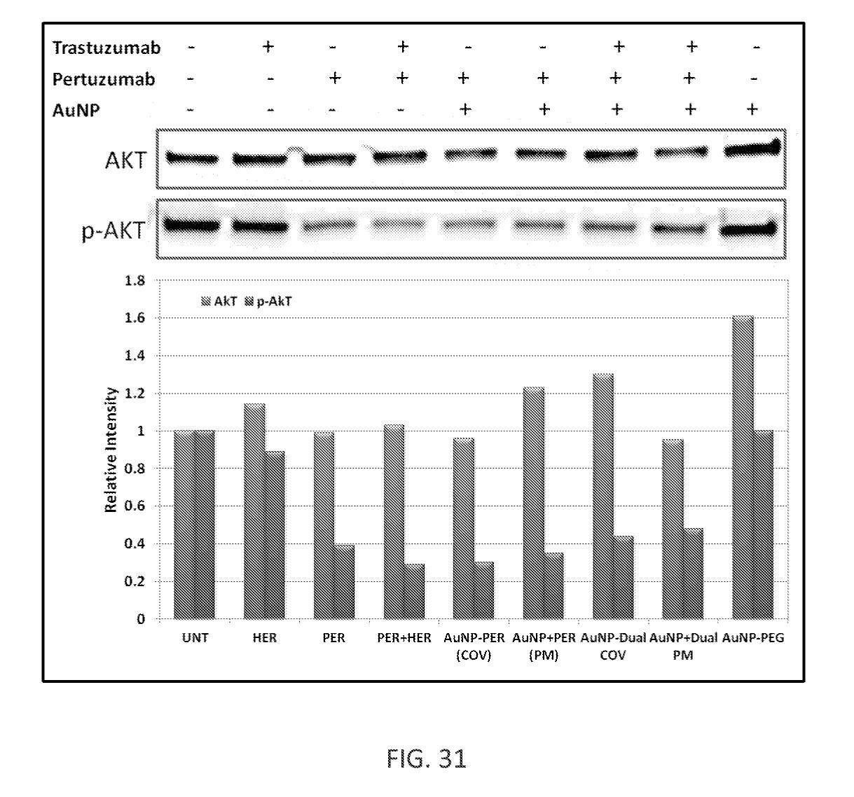

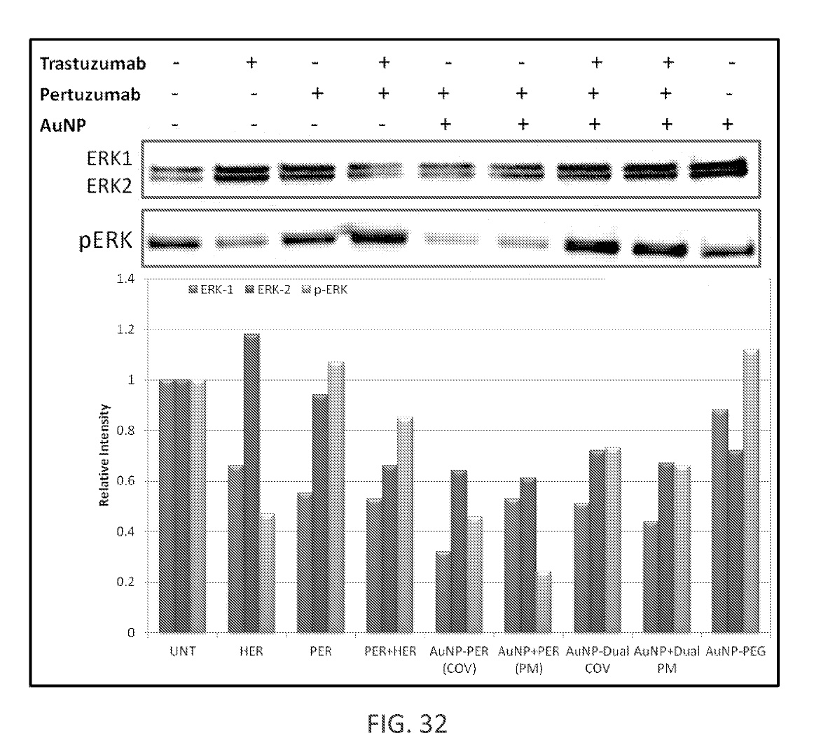

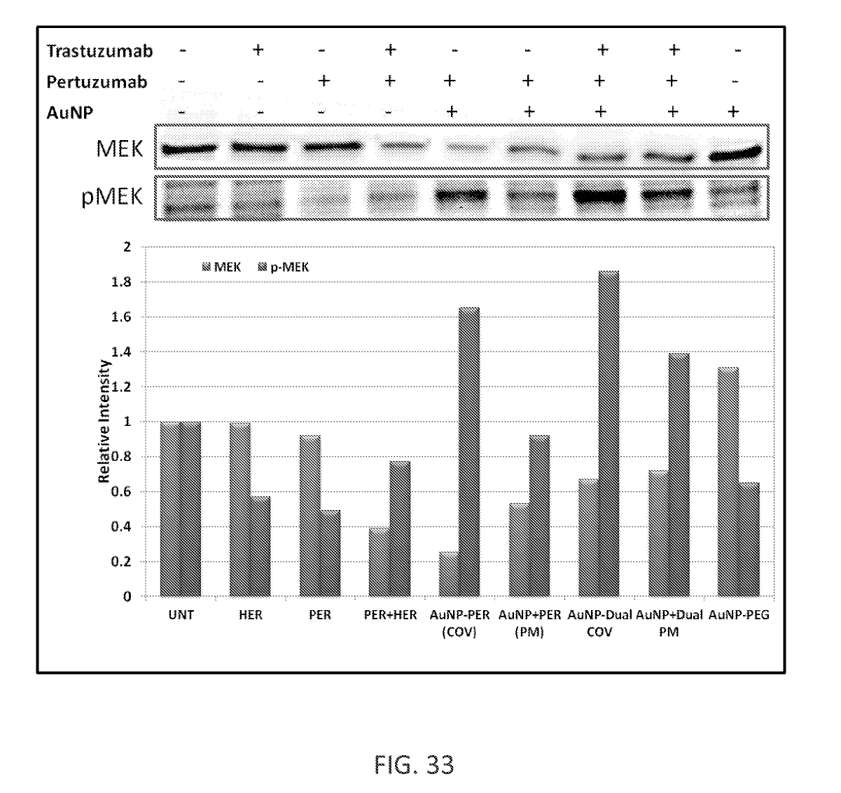

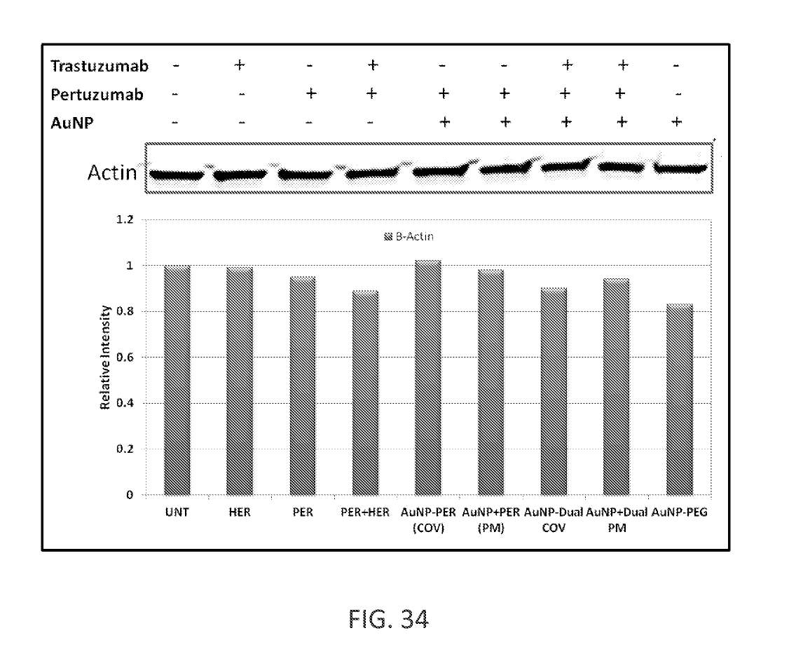

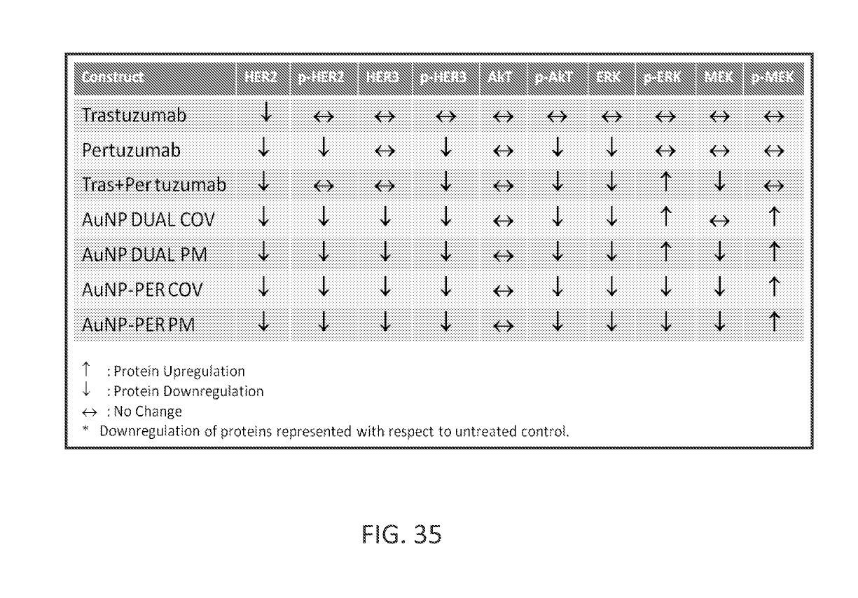

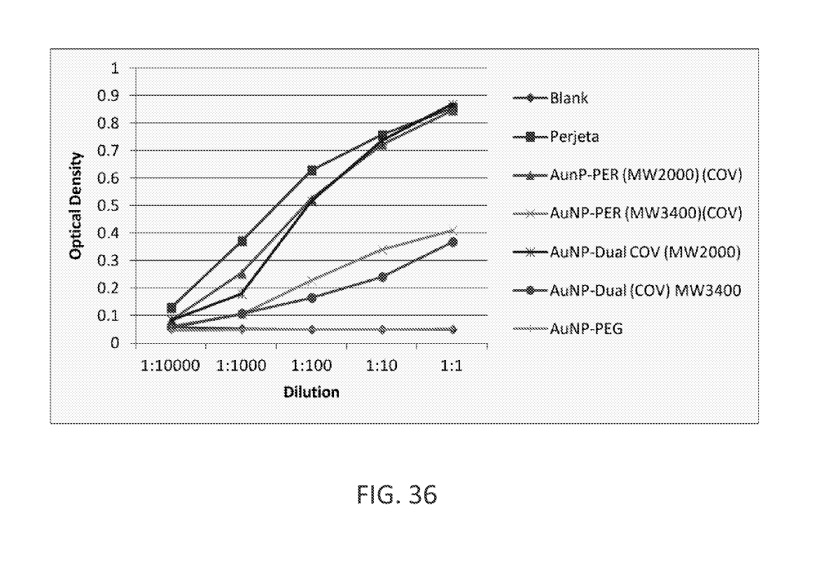

FIGS. 24-35 provide physiological characterization examples that allow determination of functionality for predetermined selection of the same. The predetermination of therapeutic function is an aspect of preferred embodiments. Linker weight determination can also be selected to optimize attachment. For example, tests showed that when molecular weight PEG linker (mol. Wt. 2000) with end terminal carboxyl groups have favorable orientation to achieve maximum availability of antibody fragments Fa and Fb. When the molecular weight or chain length of linker was increased from 2000 to 3400, the linker would form a self-coil structure making the carboxyl groups unavailable for conjugation with a particular desired antibody. ELISA binding study plots are given in FIG. 36 for antibody conjugated gold nanoparticles with 2000 and 3400 as the length of linker. Data points are given as mean absorbance. Antibody was diluted in a serial 10-fold dilution (stock of free antibody (Perjeta) used was 20 μg).

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

What is claimed is:

1) A nanoparticle conjugate comprising at least two antibody agents linked to a single nanoparticle.

2) The nanoparticle conjugate of claim 1, wherein at least one of said at least two antibody agents is linked electrostatically.

3) The nanoparticle conjugate of claim 2, wherein at least one of said at least two antibody agents is linked covalently.

4) The nanoparticle conjugate of claim 1, wherein at least one of said at least two antibody agents is linked covalently.

5) The nanoparticle conjugate of claim 1, wherein said at least two antibody agents comprise two different human antibodies.

6) The nanoparticle conjugate of claim 5, wherein at least one of said antibodies is pertuzumab.

7) The nanoparticle conjugate of claim 5, wherein at least one of said antibodies is trastuzumab.

8) The nanoparticle conjugate of claim 1, wherein said nanoparticle is a metallic nanoparticle.

9) The nanoparticle conjugate of claim 8, wherein said metallic nanoparticle is selected from a group of metals consisting of gold, platinum and palladium.

10) The nanoparticle conjugate of claim 9, wherein said metallic nanoparticle is gold.

11) The nanoparticle conjugate of claim 1, wherein said nanoparticle is a non-metallic nanoparticle.

12) The nanoparticle conjugate of claim 1, wherein at least one of said at least two antibody agents is an antibody, an antibody fragment, affibody, a peptide, a small molecule, a toxin, a recombinant humanized monoclonal antibody, a rabbit antibody, a goat antibody, a mouse antibody, or an anti-hapten antibody.

13) The nanoparticle conjugate of claim 1, comprising a linker between said nanoparticle said two antibody agents, the linker being selected from thiols and polyethylene glycols.

14) The nanoparticle conjugate of claim 14, wherein said polyethylene glycol is one of monoethylene glycol, diethylene glycol, and polyethylene glycol.

15) The nanoparticle conjugate of claim 14, wherein said thiol is at least one of thioctic acid, monothioctic acid, dithioctic acid, and trithioctic acid.

16) The nanoparticle conjugate of claim 14, wherein said linker has a molecular weight even more preferably in the range about 2000 Dalton to about 3400 Dalton.

17) The nanoparticle conjugate of claim 1, wherein the diameter of said nanoparticle conjugate is in the range of about 3 nm to 100 nm.

18) The nanoparticle conjugate of claim 17, wherein the diameter of said nanoparticle conjugate is preferably about 35 nm.

19) Use of said nanoparticle conjugate of claim 1 as a theranostic agent for cancer in humans.

20) Use of said nanoparticle conjugate of claim 1 as a theranostic agent for cancer in non-humans.

21) Use of said nanoparticle conjugate of claim 1 as a theranostic agent for at least one cancer in the list comprising, but not limited to, breast cancer, lung cancer, bone cancer, prostate cancer, and ovarian cancer.

22) Use of said nanoparticle conjugate of claim 1 as a breast cancer theranostic agent.

23) Use of said nanoparticle conjugate of claim 1 as a lung cancer theranostic agent.

24) Use of said nanoparticle conjugate of claim 1 as a bone cancer theranostic agent.

25) Use of said nanoparticle conjugate of claim 1 as a prostate cancer theranostic agent.

26) Use of said nanoparticle conjugate of claim 1 as an ovarian cancer theranostic agent.

27) A method of preparation of dual covalently or electrostatically bonded human antibody—AuNP conjugates, comprising:
preparing of citrate or Gum Arabic coated AuNPs in aqueous solution;
forming AuNP-PEG-COOH in solution;
selectively activating carboxyl terminated AuNP-PEG-COOH using EDC/Sulfo-NHS for covalent bonding or not activating for electrostatic bonding, and
simultaneously adding antibody agent 1 (Ab1) and (Ab2) to AuNP-PEG.

28) The method of claim 27, wherein Ab1 represents pertuzumab and Ab2 represents trastuzumab.

29) The method of claim 27, further comprising purifying by HPLC and isolating by SEC.

30) A method of preparation of dual electrostatically or electrostatically and covalently bonded human antibody—AuNP conjugates, comprising:
preparing citrate or Gum Arabic coated AuNPs in aqueous solution;
forming AuNP-PEG-COOH in solution;
adding a first antibody agent (Ab1) antibody to the solution containing AuNP-PEG-COOH with activation of carboxyl terminated AuNP-PEG-COOH using EDC/Sulfo-NHS for covalent or without for electrostatic;
purifying the solution by HPLC and isolating the solution by SEC; and
adding a second antibody agent (Ab2) antibody to isolated AuNP-PEG+Ab1 solution.

31) The method of claim 30, wherein Ab1 represents pertuzumab and Ab2 represents trastuzumab.

32) The method of claim 30, further comprising second purifying by HPLC and isolating by SEC.

33) A method of preparation of covalent bonded and electrostatically bonded human antibody—AuNP conjugates of the invention that is formed by sequential mixing, e.g., AuNP-PEG-Ab1+Ab2 COV ELE, comprising the steps:
i) Preparation of citrate coated AuNPs in aqueous solution;
ii) Preparation of AuNP-PEG-COOH in solution;
iii) Activation of carboxyl terminated AuNP-PEG-COOH using EDC/Sulfo-NHS;
iv) Addition of Ab1 antibody to solution containing AuNP-PEG with activated terminal groups solution;
v) Purification by HPLC, and isolation by SEC;
vi) Addition of Ab2 antibody to isolated AuNP-PEG+Ab1 solution, and
vii) Purification by HPLC.
